SEQUENCE LISTING (3 sequences)

SEQ ID NO: 1 - protein, 9 aa, Artificial Sequence
  CAR Peptide
CARSKNKDC

SEQ ID NO: 2 - protein, 7 aa, Artificial Sequence
  CAR Peptide Variant
CARSKNK

SEQ ID NO: 3 - protein, 9 aa, Artificial Sequence
  CAR Peptide Variant
CAQSNNKDC

(19) United States
(12) Patent Application Publication
(10) Pub. No.: US 2019/0231815 A1
(43) Pub. Date: Aug. 1, 2019

(54) COMPOSITIONS COMPRISING COPPER CHELATORS AND METHODS OF USE THEREOF FOR TREATING VASCULOPATHIES

(71) Applicant: INSMED INCORPORATED, Bridgewater, NJ (US)

(72) Inventor: Norbert VOELKEL, Denver, CO (US)

(21) Appl. No.: 16/311,279

(22) PCT Filed: Jul. 28, 2016
(86) PCT No.: PCT/US2016/044542

§ 371 Date: Dec. 19, 2018

Related U.S. Application Data
(60) Provisional application No. 62/293,121, filed on Feb. 9, 2016.
(60) Provisional application No. 62/198,037, filed on Jul. 28, 2015.

(51) Int. Cl. A61K 33/24; A61K 9/00; A61P 9/12; A61P 9/10; A61K 31/14

(52) U.S. Cl. CPC A61K 33/24; A61K 9/0014; A61K 31/14; A61P 9/12; A61P 9/10; A61K 9/0019

(57) ABSTRACT

The present invention relates to copper chelator compounds and their use for treating vasculopathies, for example, pulmonary hypertension (e.g., pulmonary arterial hypertension or portopulmonary hypertension). The methods include administering a composition comprising an effective amount of a copper chelator compound to a patient in need thereof. Administration can be via oral, intraperitoneal, transdermal, intravenous and inhalation routes. In another aspect of the invention, compositions are provided comprising a copper chelator compound complexed to or encapsulated by a lipid component, for example, a copper chelator encapsulated by a liposome and methods of treatment employing the same.

### CROSS REFERENCE TO RELATED APPLICATIONS

This application claims the benefit of priority to U.S. Provisional Patent Application Nos. 62/198,037, filed Jul. 28, 2015 and 62/293,121, filed Feb. 9, 2016, the disclosures of each of which are hereby incorporated by reference in their entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is INMD_123_02_WO_SeqList.txt. The text file is 1 KB, was created on Jul. 28, 2016, and is being submitted electronically via EFS-Web.

### BACKGROUND OF THE INVENTION

Vasculopathy is a general term used to describe any disease affecting blood vessels. It includes vascular abnormalities caused by degenerative, metabolic, idiopathic and inflammatory and immune system abnormalities, embolic diseases, coagulative disorders, and functional disorders such as posteri or reversible encephalopathy syndrome.

Pulmonary hypertension (PH) is one type of vasculopathy. It is characterized by an abnormally high blood pressure in the lung vasculature. It is a progressive, lethal disease that leads to heart failure and can occur in the pulmonary artery, pulmonary vein, or pulmonary capillaries. Patients experience shortness of breath, dizziness, fainting, and other symptoms, all of which are made worse by exertion. There are multiple causes, and can be of unknown origin, idiopathic, and can lead to hypertension in other systems, for example, portopulmonary hypertension in which patients have both portal and pulmonary hypertension.

Pulmonary hypertension has been classified into five groups by the World Health Organization (WHO). Group I is called pulmonary arterial hypertension (PAH), and includes PAH that has no known cause (idiopathic), inherited PAH (i.e., familial PAH or FPAH), PAH that is caused by drugs or toxins (including methamphetamine and cancer treatment agents), and PAH caused by conditions such as connective tissue diseases, HIV infection, liver disease, and congenital heart disease. Group II pulmonary hypertension is characterized as pulmonary hypertension associated with left heart disease. Group III pulmonary hypertension is characterized as PH associated with lung diseases, such as chronic obstructive pulmonary disease and interstitial lung diseases, as well as PH associated with sleep-related breathing disorders (e.g., sleep apnea). Group IV PH is PH due to chronic thrombotic and/or embolic disease, e.g., PH caused by blood clots in the lungs or blood clotting disorders. Group V includes PH caused by other disorders or conditions, e.g., blood disorders (e.g., polycythemia vera, essential thrombocythemia), systemic disorders (e.g., sarcoidosis, vasculitis), metabolic disorders (e.g., thyroid disease, glycogen storage disease)

Pulmonary arterial hypertension (PAH) afflicts approximately 200,000 people globally with approximately 30,000-40,000 of those patients in the United States. PAH patients experience constriction of pulmonary arteries and small vessel obliteration which lead to high pulmonary arterial pressures, making it difficult for the heart to pump blood to the lungs. Patients suffer from shortness of breath and fatigue which often severely limits the ability to perform physical activity.

Patients with PAH are typically treated with an endothelin receptor antagonist (ERA), phosphodiesterase type 5 (PDE-5) inhibitor, a guanylate cyclase stimulator, a prostanoid (e.g., prostacyclin), or a combination thereof. ERAs include abrisentan (Letairis®), sitaxentan, bosentan (Tracleer®), and macitentan (Opsumit®). PDE-5 inhibitors indicated for the treatment of PAH include sildenafil (Revatio®), tadalafil (Adcirca®). Prostanoids indicated for the treatment of PAH include iloprost, epoprosentol and treprostinil (Remodulin®, Tyvaso®). The one approved guanylate cyclase stimulator for PAH is riociguat (Adempas®). Additionally, patients are often treated with combinations of the aforementioned compounds.

Despite there being treatments for vasculopathies, such as PAH and portopulmonary hypertension (PPH), current therapies are associated with severe toxicity and tolerability issues. The present invention addresses these factors by providing compositions, kits and methods for treating vasculopathies.

### SUMMARY OF THE INVENTION

In one aspect, a method for treating a vasculopathy in a patient in need thereof is provided. The vasculopathy, in one embodiment, is pulmonary hypertension (e.g., pulmonary arterial hypertension (PAH) or portopulmonary hypertension (PPH)), peripheral vascular disease (PVD), ischemic lesions (e.g., lesions from critical limb ischemia (CLI)), coronary artery disease or diabetic vasculopathy. The method comprises administering to a patient in need thereof, a composition comprising an effective amount of a copper chelator compound.

Administration in one embodiment is via a pulmonary (inhalation), subcutaneous, oral, nasal, intraperitoneal (IP), or an intravenous (IV) route.

The copper chelator compound in one embodiment, is a compound of Formula (I):

XY Formula (I)
or an isomer, solvate, hydrate, deuterated analog, hydrolysis product, or a pharmaceutically acceptable salt thereof, wherein,
Y is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻², (Mo₂S₁₃)⁻², (Mo₂O₄)⁻², (Mo₂O₁₂)⁻², (Mo₂O₉)⁻², (Mo₂O₇)⁻², (Mo₂O₈)⁻², (Mo₂O₁₁)⁻², (Mo₂O₆)⁻², (Mo₂O₁₃)⁻², (MoOS₃)⁻², (MoO₂S₂)⁻², (MoO₃S)⁻², (WS₄)⁻², (W₂S₁₂)⁻², (W₂S₉)⁻², (W₂S₇)⁻², (W₂S₈)⁻², (W₂S₁₁)⁻²(W₂S₆)⁻², (W₂S₁₃)⁻², (WO₄)⁻², (W₂O₁₂)⁻², (W₂O₉)⁻², (W₂O₇)⁻², (W₂O₈)⁻², (W₂O₁₁)⁻², (W₂O₆)⁻², (W₂O₁₃)⁻², (WOS₃)⁻², (WO₂S₂)⁻², (WO₃S)⁻², or [2(OC(O)Z)]⁻²;
Z is alkyl or aryl;
X is (2Li)⁺², (2K)⁺², (2Na)⁺², Mg⁺², Ca⁺², ZN⁺², or {[N⁺(R¹) (R²) (R³) (R⁴)] [N⁺(R⁵) (R⁶) (R⁷) (R⁸)]},
R¹, R², R³, R⁵, R⁶, and R⁷ are independently H, or optionally substituted group selected from the group consisting of alkyl, alkenyl, alkynyl, aryl, heteroaryl, cycloalkyl, heterocycoalkyl, aralkyl, alkylaralkyl, heteroaralkyl, cycloalkylalkyl, and heterocycloalkylalky;
R⁴ and R⁸ are absent or independently H, or optionally substituted group selected from the group consisting of alkyl, alkenyl, alkynyl, aryl, heteroaryl, cycloalkyl, heterocycoalkyl, aralkyl, alkylaralkyl, heteroaralkyl, cycloalkylalkyl, and heterocycloalkylalkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R² and R⁴, together with N optionally forms an optionally substituted cyclic structure;
wherein R⁵ and R⁶, R⁶ and R⁷, or R⁶ and R⁸, together with N optionally forms an optionally substituted cyclic structure;
wherein R⁴ and R⁸ may be joined by a covalent bond;
wherein R¹, R², R³, R⁵, R⁶ and R⁷ are each independently optionally substituted with one or more OH, oxo, alkyl, alkenyl, alkynyl, NH₂, NHR⁹, N(R⁹)₂, —C═N(OH), or OPO₃H₂, wherein R⁹ is each independently alkyl or —C(═O)O-alkyl;
wherein R⁴ and R⁸ are each independently optionally substituted with one or more OH, oxo, alkyl, alkenyl, alkynyl, NH₂, NHR⁹, N(R⁹)₂, —C═N(OH), or —N⁺(R¹⁰)₃, wherein R¹⁰ is each independently optionally substituted alkyl; and
wherein one or more —CH₂— groups in R¹, R², R³, R⁴, R⁵, R⁶, R⁷ and R⁸ may be replaced with a moiety selected from the group consisting of O, NH, S, S(O), and S(O)₂.

The invention disclosed herein in one embodiment, encompasses the in vivo metabolic products and hydrolysis products (in vitro or in vivo) of the disclosed copper chelator compounds. Such in vivo metabolic products can result from, for example, the oxidation, reduction, hydrolysis, amidation, esterification, and the like of the administered compound, e.g., due to an enzymatic processes.

In one embodiment, Y is (MoS₄)⁻², (Mo₂S₁₂)⁻², (Mo₂S₇)⁻², (Mo₂S₈)⁻², (Mo₂S₁₁)⁻², (Mo₂S₆)⁻², (Mo₂S₁₃)⁻², (Mo₂O₄)⁻², (Mo₂O₁₂)⁻², (Mo₂O₉)⁻², (Mo₂O₇)⁻², (Mo₂O₈)⁻², (Mo₂O₁₁)⁻², (Mo₂O₆)⁻², (Mo₂O₁₃)⁻², (MoS₃)⁻², (MoO₂S₂)⁻² or (MoO₃S)⁻².

In another embodiment, Y is (WS₄)⁻², (W₂S₁₂)⁻², (W₂S₉)⁻², (W₂S₇)⁻², (W₂S₈)⁻², (W₂S₁₁)⁻², (W₂S₆)⁻², (W₂S₁₃)⁻², (WO₄)⁻², (W₂O₁₂)⁻², (W₂O₉)⁻², (W₂O₇)⁻², (W₂O₁₁)⁻², (W₂O₆)⁻², (W₂O₁₃)⁻², (WOS₃)⁻², (WO₂S₂)⁻² or (WO₃S)⁻².

In one embodiment Y is (MoS₄)⁻², (Mo₂S₁₂)⁻², (Mo₂S₉)⁻², (Mo₂S₇)⁻², (Mo₂S₈)⁻², (Mo₂S₁₁)⁻², Mo₂S₆)⁻², (Mo₂S₁₃)⁻², (WS₄)⁻², (W₂S₁₂)⁻², (W₂S₉)⁻², (W₂S₇)⁻², (W₂S₆)⁻², or (W₂S₁₃)⁻². In another embodiment, Y⁻² is (MoS₄)⁻² or (WS₄)⁻².

In another embodiment, Y is tetrathiomolybdate (TTM) (MoS₄)⁻².

In yet another embodiment, Y is trithiomolybdate (MoOS₃)⁻².

In even another embodiment, Y is dithiomolybdate (MoO₂S₂)⁻².

In one embodiment of the compound of Formula (I), X is:

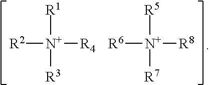

In one embodiment, [N⁺(R¹) (R²) (R³) (R⁴)]⁺ and [N⁺(R⁵) (R⁶) (R⁷) R⁸)]⁺ in {[N⁺(R¹) (R²) (R³) (R⁴)] [N⁺(R⁵) (R⁶) (R⁷) (R⁸]}⁺² can be the same or different.

In one embodiment, X is {[N⁺(R¹) (R²) (R³) (R⁴)] [N⁺(R⁵) (R⁶) (R⁷) (R⁸)]} wherein R¹, R², R³, R⁵, R⁶, and R⁷ are independently H or C₁-C₁₀ alkyl. In another embodiment, R¹, R², R³, R⁵, R⁶, and R⁷ are independently H, C₁-C₃ alkyl or C₁-C₆ alkyl. In a further embodiment, R⁴ and R⁸ are independently H or C₁-C₆ alkyl.

In one embodiment, X is {[N⁺(R¹) (R²) (R³) (R⁴)] [N⁺(R⁵) (R⁶) (R⁷) (R⁸)]} wherein R¹₁, R², R³, R⁵, R⁶, and R⁷ are independently H, methyl, ethyl or propyl. In a further embodiment, each of R¹, R², R³, R⁴, R⁵, R⁶, R⁷ and R⁸ is propyl and Y⁻² is (MoS₄)⁻², i.e., the compound is tetrapropylammoniumtetrathimolybdate. In yet another embodiment, each of R¹, R², R³, R⁴, R⁵, R⁶, R⁷ and R⁸ is methyl and Y is (MoS₄)⁻², i.e., the compound is tetramethylammoniumtetrathimolybdate. In even another embodiment, each of R¹, R², R³, R⁴, R⁵, R⁶, R⁷ and R⁸ is ethyl and Y is (MoS₄)⁻², i.e., the compound is tetraethylammoniumtetrathimolybdate.

In one embodiment, X is {[N⁺(R¹) (R²) (R³) (R⁴)] [N⁺(R⁵) (R⁶) (R⁷) (R⁸)]} wherein R¹, R², R³, R⁵, R⁶, and R⁷ are independently methyl; and R⁴ and R⁸ is each optionally substituted alkyl; and Y is (MoS₄)⁻², i.e., the compound is tetramethylammoniumtetrathimolybdate. In yet another embodiment, each of R¹, R², R³, R⁵, R⁶, and R⁷ are independently methyl; R⁴ and R⁸ is each optionally substituted ethyl; and Y is (MoS₄)⁻², i.e., the compound is tetramethylammoniumtetrathimolybdate. In a further embodiment, each of R¹, R², R³, R⁵, R⁶, and R⁷ are independently methyl; R⁴ and R⁸ is each substituted ethyl, wherein the substituent is a hydroxyl; and Y is (MoS₄)⁻², i.e., the compound is tetramethylammoniumtetrathimolybdate. In one embodiment, each of R¹, R², R³, R⁵, R⁶, and R⁷ are independently methyl; R⁴ and R⁸ is each —CH₂CH₂—OH; and Y is (MoS₄)⁻², i.e., the compound is tetramethylammonium tetrathimolybdate.

In one embodiment, the copper chelator compound of Formula (I) is a bis-choline tetrathiomolybdate.

In one embodiment, the copper chelator compound of Formula (I) is:

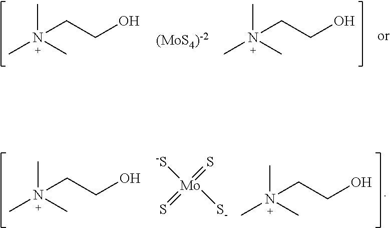

In one embodiment, X is (2Na)⁺² and Y is (MoS₄)⁻².

In one embodiment, the copper chelator compound is a compound of Formula (II):

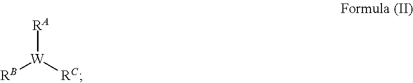

or a deprotonated anion, isomer, deuterated analog, solvate, hydrate, hydrolysis product or a pharmaceutically acceptable salt thereof, wherein,

W is N, O, or S;

R^{A}, R^{B}, and R^{C} are each independently H, alkyl, aryl, heteroaryl, cycloalkyl, heterocycoalkyl, aralkyl, alkylaralkyl, heteroaralkyl, cycloalkylalkyl, or heterocycloalkylalkyl, provided that when W is O, or S, R^{C} is absent;
wherein when R^{A}, R^{B}, and/or R^{C} are alkyl, one or more carbon atoms of alkyl may be replaced with O, NH, NR¹¹, S, S(O), and S(O)₂, provided that no two adjacent carbon atoms are replaced with heteroatoms, wherein R¹¹ is each independently alkyl, -alkyl-COOH, —OC(O)alkyl, aryl, heteroaryl, cycloalkyl, heterocycoalkyl, aralkyl, alkylaralkyl, heteroaralkyl, cycloalkylalkyl, or heterocycloalkylalkyl,
wherein R^{A} and R^{B} together with W may form an optionally substituted cyclic structure comprising 5 to 30 atoms in the ring, wherein one or more carbon atoms in the ring may be replaced with a heteroatom selected from the group consisting of O, NH, NR¹¹, S, S(O), and S(O)₂, provided that no two adjacent carbon atoms are replaced with heteroatoms;
wherein two R¹¹ may join to form an optionally substituted cyclic structure comprising 5 to 30 atoms in the ring, wherein one or more carbon atoms in the ring may be replaced with a heteroatom selected from the group consisting of O, NH, S, S(O), and S(O)₂, provided that no two adjacent carbon atoms are replaced with heteroatoms;
wherein R^{A}, R^{B} and R^{C} are optionally substituted with one or more halogen, —OH, —SH, —COOH oxo, alkyl, alkenyl, alkynyl, NH₂, NHR⁹, N(R⁹)₂, —C═N(OH), or OPO₃H₂, wherein R⁹ is each independently alkyl, —C(═O)O-alkyl, —C(═O)-alkyl, aryl, heteroaryl, aralkyl, or heteroarylalkyl;
wherein the deprotonated anion of Formula (II) indicates that one or more H⁺ from OH or SH has been removed to provide O⁻ or S⁻, respectively.

In one embodiment of a compound of Formula (II), R^{A}, R^{B}, and R^{C} are each independently H or optionally substituted alkyl, heteroaryl, aryl, aralkyl, or heteroarylalkyl. In another embodiment, R^{A}, R^{B}, and R^{C} are each independently H or optionally substituted pyridine, —C₁-C₃ alkyl-pyridine, or —C₁-C₃ alkyl-phenyl.

In another aspect of the invention, a pharmaceutical composition is provided comprising a copper chelator compound complexed to or encapsulated by a lipid component. The lipid component in one embodiment is present in liposomes. In a further embodiment, the lipid component comprises a phospholipid. In even a further embodiment, the phospholipid is a negatively charged phospholipid such as a phosphatidylglycerol (PG) or a phosphatidylserine (PS). In yet another embodiment, the phospholipid is a phosphatidylcholine (PC), phosphatidylglycerol (PG), phosphatidylinositol (PI), phosphatidylserine (PS), phosphatidylethanolamine (PE), phosphatidic acid (PA) or a combination thereof. The lipid component in a further embodiment, include a sterol, for example, cholesterol.

In yet another aspect of the invention, a pharmaceutical composition is provided comprising a copper chelator compound and a polymeric material is provided. The polymeric material in one embodiment is a water swellable polymer, a hydrophilic polymer, a hydrophobic polymer or a combination thereof. For example, the polymer can be a polysaccharide, hydrogel, methyl cellulose, hydroxypropyl methyl cellulose, hydroxypropyl cellulose, hydroxyethyl cellulose, nitro cellulose, carboxymethyl cellulose, a cellulose ether, a polyethylene oxide or a combination thereof. In one embodiment, the composition is formulated as nanoparticles.

Yet another aspect of the invention comprises a kit for the treatment of a vasculopathy. The kit in one embodiment comprises a copper chelator composition, together with an inhalation delivery device, a subcutaneous infusion pump or an intravenous infusion pump. In one embodiment, the inhalation delivery device is a dry powder inhaler (DPI), metered dose inhaler (MDI), soft mist inhaler, or a nebulizer.

### DETAILED DESCRIPTION OF THE INVENTION

Pulmonary arterial hypertension (PAH) constitutes a group of orphan diseases that are characterized by high pulmonary artery pressure—eventually leading to fatal right heart failure. Histological examination of the lungs of such PAH patients show that millions of small lung vessels (arterioles in the periphery of the lung) are obliterated by cells that grow and fill the lumen of these vessels. These cells are abnormal—they have a phenotype that is apoptosis-resistant.

Presently used drugs f©r “targeted” PAH therapy are without exception vasodilators. In contrast, the compounds, compositions and methods described herein, without wishing to be bound by theory, treat vaculopathies such as PAH by re-opening obliterated vasculature.

Copper is angiogenic, which means vessel growth and the growth of vascular lining cells (endothelial cells) is highly copper-dependent. By chelating and removing copper (and by molybdenum or tungsten effecting a steric hindrance of copper in the catalytic center of copper-dependent enzymes) from the abnormally growing endothelial cells that obliterate the lumen of arterioles in the lungs of PAH patients, two disease-modifying aspects are combatted (i) separation of these abnormal cells from their matrix which will cause their death (referred to in the art as “anoikis”). Cells cannot survive in isolation; they must sit on a basement membrane or matrix and (ii) the propagation of differentiation of vascular stem cells in the vessel wall in the setting of vascular wall injury. Accordingly, it is thought that the present invention fulfills a need in the treatment of PAH by (i) killing abnormal, lumen filling cells and (ii) by normalizing stem cells (achieving their differentiation to a normal vessel lung cell, thereby opening arterioles and terminating the out-of-control wound healing process.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Throughout the present specification, the terms “about” and/or “approximately” can be used in conjunction with numerical values and/or ranges. The term “about” is understood to mean those values near to a recited value. For example, “about 40 [units]” can mean within ±25% of 40 [units](e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” can be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

“Amino” refers to the —NH₂ radical.

“Halo” or “halogen” refers to bromo, chloro, fluoro or iodo radical.

“Hydroxy” or “hydroxyl” refers to the OH radical.

“Imino” refers to the ═NH substituent.

“Nitro” refers to the NO₂ radical.

“Oxo” refers to the ═O substituent.

The term “alkyl” or “alkyl group” refers to a monovalent, fully saturated, straight or branched hydrocarbon chain radical which is attached to the rest of the molecule by a single bond. Alkyls comprising any number of carbon atoms from 1 to 30 are included, wherein alkyl chain length is indicated by a range of numbers, and a branched alkyl, wherein a branching point in the chain exists, and the total number of carbons in the chain is indicated by a range of numbers. For example, an alkyl comprising up to 16 carbon atoms is a C₁-C₁₆ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉, and C₁₀ alkyls. Similarly, a C₁-C₁₂ alkyl includes all the foregoing moieties, but also includes C₁₁ and C₁₂ alkyls, Non-limiting examples of C₁-C₁₆ alkyl include methyl, ethyl, n-propyl, sec-propyl, n-butyl, sec-butyl, t-butyl, n-pentyl, t-amyl, n-hexyl, n-heptyl, n-octyl, n-nonyl, n-decyl, n-undecyl, and n-dodecyl, hexadecyl, heptadecyl, octadecyl. Unless stated otherwise specifically, an alkyl group can be optionally substituted.

The term “alkylene” or “alkylene chain” refers to a fully saturated, straight or branched divalent hydrocarbon chain radical, and having from 1 to 30 carbon atoms. Non-limiting examples of alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically, an alkylene chain can be optionally substituted.

The term “alkenyl” or “alkenyl group” refers to a monovalent, straight or branched hydrocarbon chain radical having from 2 to 30 carbon atoms, and having one or more carbon-carbon double bonds. Each alkenyl group is attached to the rest of the molecule by a single bond. For example, an alkenyl group comprising up to 16 carbon atoms is a C₂-C₁₆ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically, an alkenyl group can be optionally substituted.

The term, “alkenylene” or “alkenylene chain” refers to a straight or branched divalent hydrocarbon chain radical, having from 2 to 30 carbon atoms, and having one or more carbon-carbon double bonds. Non-limiting examples of alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically, an alkenylene chain can be optionally substituted.

The term “alkynyl” or “alkynyl group” refers to a monovalent, straight or branched hydrocarbon chain radical having from 2 to 30 carbon atoms, and having one or more carbon-carbon triple bonds. Each alkynyl group is attached to the rest of the molecule by a single bond. For example, an alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of alkynyl include ethynyl, propynyl, butynyl, pentynyl and the like. Unless stated otherwise specifically, an alkyl group can be optionally substituted.

The term “alkynylene” or “alkynylene chain” refers to a straight or branched divalent hydrocarbon chain radical, having from 2 to 30 carbon atoms, and having one or more carbon-carbon triple bonds. Non-limiting examples of alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically, an alkynylene chain can be optionally substituted.

The term “alkoxy” refers to a radical of the formula ORₐ where Rₐ is an alkyl, alkenyl or alknyl radical as defined above containing one to twelve carbon atoms. Unless stated otherwise specifically, an alkoxy group can be optionally substituted.

The term “alkylamino” refers to a radical of the formula —NHRa or —NRₐRₐ where each Ra is, independently, an alkyl, alkenyl or alkynyl radical as defined above containing one to twelve carbon atoms. Unless stated otherwise specifically, an alkylamino group can be optionally substituted.

The term “alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetyl”) moiety. Alkylcarbonyl groups can also be referred to as “Cᵥ-C_{z}, acyl” where v and z depicts the range of the number of carbon in Rₐ, as defined above. For example, “C₁-C₁₀ acyl” refers to alkylcarbonyl group as defined above, where Rₐ is C_{I}-C₁₀ alkyl, C₁-C₁₀ alkenyl, or C₁-C₁₀ alkynyl radical as defined above. Unless stated otherwise specifically, an alkyl carbonyl group can be optionally substituted.

The term “aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically, the term “aryl” is meant to include aryl radicals that are optionally substituted.

The terms “aralkyl” or “arylalkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically, an aralkyl group can be optionally substituted.

The term “carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyls, cycloalkenyls and cycloalkynyls as defined herein. Unless stated otherwise specifically, a carbocyclyl group can be optionally substituted.

The term “cycloalkyl” refers to a stable non aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically, a cycloalkyl group can be optionally substituted.

The term “cycloalkenyl” refers to a stable non aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically, a cycloalkenyl group can be optionally substituted.

The term “cycloalkynyl” refers to a stable non aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically, a cycloalkynyl group can be optionally substituted.

The term “cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically, a cycloalkylalkyl group can be optionally substituted.

The term “haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically, a haloalkyl group can be optionally substituted.

The term “haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically, a haloalkenyl group can be optionally substituted.

The term “haloalkynyl” refers to an alkynyl radical, as defined above, which is substituted by one or more halo radicals, as defined above, e.g., 1-fluoropropynyl, 1-fluorobutynyl, and the like. Unless stated otherwise specifically, a haloalkenyl group can be optionally substituted.

The term “heterocyclyl,” “heterocyclic ring” or “heterocycle” refers to a stable 3 to 20 membered non aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo thiomorpholinyl. Unless stated otherwise specifically, a heterocyclyl group can be optionally substituted.

The term “N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically, an N-heterocyclyl group can be optionally substituted.

The term “heterocyclylalkyl” refers to a radical of the formula —R_{b}—Rₑ where R_{b} is an alkylene, alkenylene, or alkynylene chain as defined above and Rₑ is a heterocyclyl radical as defined above, and if the heterocyclyl is a nitrogen containing heterocyclyl, the heterocyclyl can be attached to the alkyl, alkenyl, and alkynyl radical at the nitrogen atom. Unless stated otherwise specifically, a heterocyclylalkyl group can be optionally substituted.

The term “heteroaryl” refers to a 5 to 20 membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2 a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1 oxidopyrimidinyl, 1-oxidopyrazinyl, 1-oxidopyridazinyl, 1 phenyl 1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically, a heteroaryl group can be optionally substituted.

The term “N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically, an N-heteroaryl group can be optionally substituted.

The terms “heteroaralkyl” or “heteroarylalkyl” refers to a radical of the formula —R_{b}—R_{f} where R_{b} is an alkylene, alkenylene, or alkynylene chain as defined above and R_{f} is a heteroaryl radical as defined above. Unless stated otherwise specifically, a heteroarylalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ—NR_{g}SO₂Rₕ, —OC(═O)NR_{g} Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

The term “fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

The terms “optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R) or (S) or, as (D) or (L) for amino acids. The present invention is meant to include all such possible isomers, as well as their racemic and optically pure forms whether or not they are specifically depicted herein. Optically active (+) and (−), (R) and (S), or (D) and (L) isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The term “isomer” as used herein, refers to stereoisomers, diastereomers, enantiomers, constitutional isomers, tautomers, and the like.

The term “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

The term “tautomer” refers to a proton shift from one atom of a molecule to another atom of the same molecule. The present invention includes tautomers of any said compounds.

The term “deprotonated anion” as used herein, refers to an anion resulted from removing one or more of H from —OH (including —COOH) or —SH groups in a molecule resulting in a negatively charged —O⁻ or —S⁻ species, respectively. Deprotonated anion can have a negative charge of −1, −2, −3, or −4.

The terms “deuterated” or “deuterated analog” refers to a compound where at least one H has been replaced with D (deuterium). Ina deuterated compound, deuterium is present in at least 100 times the natural abundance level. Unless stated otherwise specifically, any compound of this disclosure may be deuterated in one or more positions.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 software naming program (CambridgeSoft). For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for sonic carbon atoms, which are assumed to be bonded to sufficient hydrogen atoms to complete the valency.

The term “pharmaceutically acceptable carrier, diluent or excipient” includes without limitation any adjuvant, earlier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “pharmaceutically acceptable salt” includes both acid and base addition salts.

The term “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

The term “pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropyl amine, diethanol amine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

The term “pharmaceutical composition” refers to a formulation of a compound of the invention and a medium generally accepted in the art for the delivery of the biologically active compound to a mammal, e.g., a human. Such a medium includes all pharmaceutically acceptable carriers, diluents or excipients therefor.

The term “effective amount” refers to a therapeutically effective amount or a prophylactically effective amount. A “therapeutically effective amount” refers to an amount effective, at dosages and for periods of time necessary, to achieve a desired therapeutic result. A therapeutically effective amount of a compound can vary according to factors such as the disease state, age, sex, and weight of the subject, and the ability of the compound to elicit a desired response in the subject. Dosage regimens can be adjusted to provide an optimum therapeutic response. A therapeutically effective amount is also one in which any toxic or detrimental effects of the compound are outweighed by therapeutically beneficial effects. A “prophylactically effective amount” refers to an amount effective, at dosages and for periods of time necessary, to achieve a desired prophylactic result.

One aspect of the present invention provides a composition comprising an effective amount of a copper chelator compound. The composition can be used as described herein, to treat a vasculopathy in a patient in need thereof. The vasculopathy in one embodiment is pulmonary arterial hypertension (PAH) or portopulmonary hypertension (PPH).

In one embodiment, the copper chelator is a compound of Formula (I):

XY Formula (I)
or an isomer, solvate, hydrate, deuterated analog, hydrolysis product, or a pharmaceutically acceptable salt thereof, wherein,
Y is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻², (Mo₂S₁₃)⁻², (MoO₄)⁻², (Mo₂O₁₂)⁻², (Mo₂O₉)⁻², (Mo₂O₇)⁻², (Mo₂O₈)⁻², (Mo₂O₁₁)⁻², (Mo₂O₆)⁻², (Mo₂O₁₃)⁻², (MoOS₃)⁻², (MoO₂S₂)⁻², (MoO₃S)⁻², (WS₄)⁻², (W₂S₁₂)⁻², (W₂S₉)⁻², (W₂S₇)⁻², (W₂S₈)⁻², (W₂S₁₁)⁻²(W₂S₆)⁻², (W₂S₁₃)⁻², (WO₄)⁻², (W₂O₁₂)⁻², (W₂O₉)⁻², (W₂O₇)⁻², (W₂O₈)⁻², (W₂O₁₁)⁻², (W₂O₆)⁻², (W₂O₁₃)⁻², (WOS₃)⁻², (WO₂S₂)⁻², (WO₃S)⁻², or [2(OC(O)Z)]⁻²;
Z is alkyl or aryl;
X is (2Li)⁺², (2K)⁺², (2Na)⁺², Mg⁺², Ca⁺², ZN⁺², or {[N⁺(R¹) (R²) (R³) (R⁴)] [N⁺(R⁵) (R⁶) (R⁷) (R⁸)]},
R¹, R², R³, R⁵, R⁶, and R⁷ are independently H, or optionally substituted group selected from the group consisting of alkyl, alkenyl, alkynyl, aryl, heteroaryl, cycloalkyl, heterocycoalkyl, aralkyl, alkyl aralkyl, heteroaralkyl, cycloalkylalkyl, and heterocycloalkylalky;
R⁴ and R⁸ are absent or independently H, or optionally substituted group selected from the group consisting of alkyl, alkenyl, alkynyl, aryl, heteroaryl, cycloalkyl, heterocycoalkyl, aralkyl, alkylaralkyl, heteroaralkyl, cycloalkylalkyl, and heterocycloalkylalkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R² and R⁴, together with N optionally forms an optionally substituted cyclic structure;

wherein R⁵ and R⁶, R⁶ and R⁷, or R⁶ and R⁸, together with N optionally forms an optionally substituted cyclic structure;
wherein R⁴ and R⁸ are optionally joined by a covalent bond;
wherein R¹, R², R³, R⁵, R⁶ and R⁷ are each independently optionally substituted with one or more OH, oxo, alkyl, alkenyl, alkynyl, NH₂, NHR⁹, N(R⁹)₂, —C═N(OH), or OPO₃H₂, wherein R⁹ is each independently alkyl or —C(═O)O-alkyl;
wherein R⁴ and R⁸ are each independently optionally substituted with one or more OH, oxo, alkyl, alkenyl, alkynyl, NH₂, NHR⁹, N(R⁹)₂, —C═N(OH), or —N(R¹⁰)₃, wherein R¹⁰ is each independently optionally substituted alkyl; and
wherein one or more —CH₂— groups in R¹, R², R³, R⁴, R⁵, R⁶, R⁷ and R⁸ is optionally replaced with a moiety selected from the group consisting of O, NH, S, S(O), and S(O)₂.

The invention disclosed herein in one embodiment, encompasses the in vivo metabolic products and hydrolysis products (in vitro or in vivo) of the disclosed copper chelator compounds. Such in vivo metabolic products can result from, for example, the oxidation, reduction, hydrolysis, amidation, esterification, and the like of the administered compound, primarily due to enzymatic processes.

In one embodiment, X is (2Na)⁺² and Y is (MoS₄)⁻².

In one embodiment, Y is (MoS₄)⁻², (Mo₂S₁₂)⁻², (Mo₂S₉)⁻²(Mo₂S₇)⁻², (Mo₂S₈)⁻², (Mo₂S₁₁)⁻², (Mo₂S₆)⁻², (Mo₂S₁₃)⁻², (MoO₄)⁻², (Mo₂O₁₂)⁻²(Mo₂O₉)⁻², (Mo₂O₇)⁻², (Mo₂O₈)⁻², (Mo₂O₁₁)⁻², (Mo₂O₆)⁻², (Mo₂O₁₃)⁻², (MoOS₃)⁻², (MoO₂S₂S₂)⁻², or (MoO₃S)⁻².

In another embodiment, Y is (WS₄)⁻², (W₂S₁₂)⁻², (W₂S₉)⁻², (W₂S₇)⁻², (W₂S₈)⁻²(W₂S₈)⁻², (W₂S₁₁)⁻², (W₂S₆)⁻², (W₂S₁₃)⁻², (WO₄)⁻², (W₂O₁₂)⁻², (W₂O₉)⁻², (W₂O₇) ⁻²(W₂O₈)⁻², (W₂O₁₁)⁻², (W₂O₆)⁻², (W₂O₁₃)⁻², (WOS₃)⁻², (WO₂S₂)⁻², or (WO₃S₂)⁻².

In one embodiment, Y is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻², (Mo₂S₁₃)⁻², (WS₄)⁻², (W₂S₁₂)⁻², (W₂S₉)⁻², (W₂S₇)⁻², (W₂S₈)⁻², (W₂S₁₁)⁻², (W₂S₆)⁻², or (W₂S₁₃)⁻². In another embodiment, Y⁻² is (MoS₄)⁻² or (WS₄)⁻².

In another embodiment, Y is tetrathiomolybdate (TTM) (MoS₄)⁻².

In yet another embodiment, Y is trithiomolybdate (MoOS₃)⁻².

In even another embodiment, Y is dithiomolybdate (MoO₂S₂)⁻².

In one embodiment, X in Formula (I) is

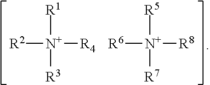

In one embodiment, [N⁺(R¹) (R²) (R³) (R⁴)]⁺ and [N^{-H}(R⁵) (R⁶) (R⁷) (R^{s})] in {[N⁺(R¹) (R²) (R³) (R⁴)] [N⁺ (R⁵) (R⁶) (R⁷) (R⁸)]} ⁺² are the same or different.

In one embodiment, X is {[N⁺(R¹) (R²) (R³) (R⁴)] [N⁺ (R⁵) (R⁶) (R⁷) (R⁸)]} wherein R¹, R², R³, R⁵, R⁶, and R⁷ are independently H or C₁-C₁₀ alkyl. In another embodiment, R¹, R² R³, R⁵, R⁶, and R⁷ are independently H, C₁-C₃ alkyl or Cᵢ-C₆ alkyl. In a further embodiment, R⁴ and R⁸ are independently H or C₁-C₆ alkyl.

In one embodiment, X is {[N⁺ (R¹) (R²) (R³) (R⁴)] [N⁺ (R⁵) (R⁶) (R⁷) (R⁸)]} wherein R¹, R², R³, R⁵, R⁶, and R⁷ are independently H, methyl, ethyl or propyl. In a further embodiment, each of R¹, R², R³, R⁴, R⁵, R⁶, R⁷ and R⁸ is propyl and Y⁻² is (MoS₄)⁻², i.e., the compound is tetrapropylammoniumtetrathimolybdate. In yet another embodiment, each of R¹, R², R³, R⁴ R⁵, R⁶, R⁷ and R⁸ is methyl and Y is (MoS₄)⁻², i.e., the compound is tetramethylammoniumtetrathimolybdate. In even another embodiment, each of R¹, R², R³, R⁴, R⁵, R⁶, R⁷ and R⁸ is ethyl and Y is (MoS₄)⁻², i.e., the compound is tetraethylammoniumtetrathimolybdate.

In one embodiment, X is {[N⁺(R¹) (R²) (R³) (R⁴)] [N⁺ (R⁵) (R⁶) (R⁷) (R⁸)]} wherein R¹, R² and R³ are independently H, methyl, or ethyl and R⁴ is H or an optionally substituted alkyl, alkenyl, cycloalkylalkyl, cycloalkyl, aryl, aralkyl, heterocycloalkyl, or heteroaryl. In another embodiment. X is {[N⁺(R¹) (R²) (R³) (R⁴)] [N⁺ (R⁵) (R⁶) (R⁷) (R⁸)]} wherein R⁵, R⁶, and R⁷ are independently H, methyl, or ethyl and R⁸ is H or an optionally substituted alkyl, alkenyl, cycloalkylalkyl, cycloalkyl, aryl, aralkyl, heterocycloalkyl, or heteroaryl. In one embodiment, the optional substituents for R⁴ and/or R⁸ are selected from the group consisting of alkyl, OH, NH₂, and oxo. In another embodiment, one or more —CH₂— groups of R⁴ and/or R⁸ are replaced with a moiety selected from O, NH, S, S(O), and S(O)₂.

In one embodiment, X is {[N⁺(R¹) (R²) (R³) (R⁴)] [N⁺ (R⁵) (R⁶) (R⁷) (R⁸)]} wherein R¹, R², R³, R⁵R⁶, and R⁷ are independently methyl and R⁴ and R⁸ is each optionally substituted alkyl. In yet another embodiment, X is {[N⁺(R¹) (R²) (R³) (R⁴)] [N⁺(R⁵) (R⁶) (R⁷) (R⁸)]} wherein each of R¹, R², R³, R⁵, R^{s}, and R⁷ are independently methyl and R⁴ and R⁸ is each optionally substituted ethyl. In a further embodiment, X is {[N⁺(R¹) (R²) (R³) (R⁴)] [N⁺ (R⁵) (R⁶) (R⁷) (R⁸)]} wherein each of R¹, R², R³, R⁵, R⁶, and R⁷ are independently methyl and R⁴ and R⁸ is each substituted ethyl, wherein the substituent is a hydroxyl. In one embodiment, X is {[N⁺(R¹) (R²) (R³) (R⁴)] [N⁺ (R⁵) (R⁶) (R⁷) (R⁸)]} wherein each of R¹, R², R³, R⁵, R⁶, and R⁷ are independently methyl and R⁴ and R⁸ is each —CH₂CH₂—OH.

In one embodiment, X is {[N⁺ (R¹) (R²) (R³) (R⁴)] [N⁺ (R⁵) (R⁶) (R⁷) (R⁸)]} wherein R¹, R², R³, R⁵, R⁶, and R⁷ are independently methyl; R⁴ and R⁸ is each optionally substituted alkyl; and Y is (MoS₄)⁻², i.e., the compound is tetramethylammoniumtetrathimolybdate. In yet another embodiment, X is {[N⁺ (R¹) (R²) (R³) (R⁴)] [N⁺ (R⁵) (R⁶) (R⁷) R⁸)]} wherein each of R¹, R², R³, R⁵, R⁶, and R⁷ are independently methyl; R⁴ and R⁸ is each optionally substituted ethyl; and Y is (MoS₄)⁻², i.e., the compound is tetramethylammoniumtetrathimolybdate. In a further embodiment, X is {[N⁺ (R¹) (R²) (R³) (R⁴)] [N⁺ (R⁵) (R⁶) (R⁷) R⁸)]} wherein each of R¹, R², R³, R⁵, R⁶, and R⁷ are independently methyl; R⁴ and R⁸ is each substituted ethyl, wherein the substituent is a hydroxyl; and Y is (MoS₄)⁻², i.e., the compound is tetramethylammoniumtetrathimolybdate. In one embodiment, X is {[N⁺ (R¹) (R²) (R³) (R⁴)] [N⁺ (R⁵) (R⁶) (R⁷) R⁸)]} wherein each of R^{I}, R², R³, R⁵, R⁶, and R⁷ are independently methyl; R⁴ and R⁸ is each —CH₂CH₂—OH; and Y is (MoS₄)⁻², i.e., the compound is tetramethylammoniumtetrathimolybdate.

In one embodiment, the copper chelator compound of Formula (I) is a bis-choline tetrathiomolybdate.

In one embodiment, the copper chelator compound of Formula (I) is:

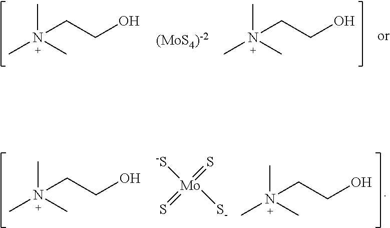

Table 1 provides non-limiting embodiments of {[N⁺ (R¹) (R²) (R³) (R⁴)] [N⁺ (R⁵) (R⁶) (R⁷) (R⁸)]}.

TABLE 1
Non-limiting embodiments of {[N⁺(R¹)(R²)(R³)(R⁴)][N⁺(R⁵)(R⁶)(R⁷)(R⁸)]}

| | R¹ | R² | R³ | R⁴ | R⁵ | R⁶ | R⁷ | R⁸ |
|---|---|---|---|---|---|---|---|---|
| 1 | H | H | H | H | H | H | H | H |
| 2 | CH₃ | CH₃ | CH₃ | CH₃ | CH₃ | CH₃ | CH₃ | CH₃ |
| 3 | ethyl | ethyl | ethyl | ethyl | ethyl | ethyl | ethyl | ethyl |
| 4 | propyl | propyl | propyl | propyl | propyl | propyl | propyl | propyl |
| 5 | butyl | butyl | butyl | butyl | butyl | butyl | butyl | butyl |
| 6 | pentyl | pentyl | pentyl | pentyl | pentyl | pentyl | pentyl | pentyl |
| 7 | H | H | H | H | CH₃ | CH₃ | CH₃ | CH₃ |
| 8 | H | H | H | H | ethyl | ethyl | ethyl | ethyl |
| 9 | H | H | H | H | propyl | propyl | propyl | propyl |
| 10 | H | H | H | H | butyl | butyl | butyl | butyl |
| 11 | CH₃ | CH₃ | CH₃ | CH₃ | ethyl | ethyl | ethyl | ethyl |
| 12 | CH₃ | CH₃ | CH₃ | CH₃ | propyl | propyl | propyl | propyl |
| 13 | CH₃ | CH₃ | CH₃ | CH₂CH₂OH | CH₃ | CH₃ | CH₃ | CH₂CH₂OH |

ethyl = CH₂CH₃

In one embodiment, [N⁺ (R¹) (R²) (R³) (R⁴)] and/or [N⁺ (R⁵) (R⁶) R⁷) (R⁸)] in {[N⁺(R¹) (R²) (R³) (R⁴)] [N⁺ (R⁵) (R⁶) (R⁷) (R⁸)]} is independently:

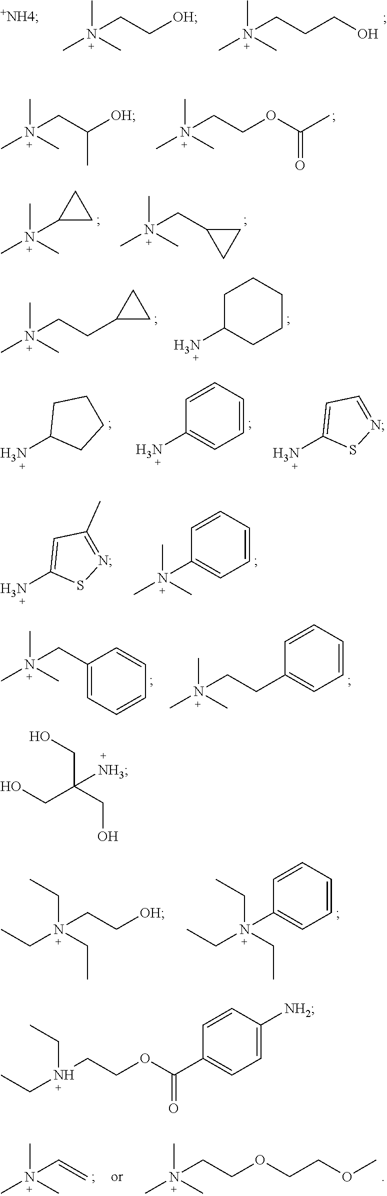

In one embodiment, at least one of [N⁺ (R¹) (R²) (R³) (R⁴)] and [N⁺(R⁵) (R⁶) (R⁷) R⁸)] in {[N(R¹) R²) (R³) (R⁴)] [N⁺ (R⁵) R⁶) (R⁷) (R⁸)]} is:

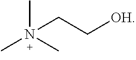

In another embodiment, [N⁺ (R¹) (R²) (R³) (R⁴)] and [N⁺ (R⁵) (R⁶) R⁷) (R⁸)] in {[N⁺(R¹) (R²) (R³) R⁴)] [N⁺ (R⁵) (R⁶) (R⁷) (R⁸)]} are each

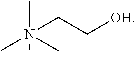

In one embodiment, X is {[N⁺(R¹) (R²) (R³) (R⁴)] [N⁺ (R⁵) (R⁶) (R⁷) (R⁸)]}wherein R¹, R², R³ and R⁴ in [N⁺ (R¹) (R²) (R³) (R⁴)] are each independently H or alkyl. In another embodiment, X is {[N⁺(R¹) (R²) (R³) (R⁴)] [N⁺(R⁵) (R⁶) (R⁷) (W)]} wherein R⁵, R⁶, R⁷ and R⁸ in [N⁺ (R⁵) (R⁶) (R⁷) (R⁸)] are each independently H or alkyl.

In one embodiment, X is {[N⁺(R¹) (R²) (R³) (R⁴)] [N⁺(R⁵) (R⁶) (R⁷) (R⁸)]} wherein R⁴ and R⁸ are joined by a covalent bond. For example, if R₄ and R₈ are both methyl, when R₄ and R₈ are joined by a covalent bond, it can form an ethylene link between the two nitrogens as illustrated below:

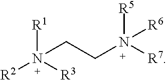

In one embodiment, X is {[N⁺(R¹) (R²) (R³) (R⁴)] [N⁺(R⁵) (R⁶) (R⁷) (R⁸)]} wherein R⁴ and R⁸ are both optionally substituted alkyl group joined by a covalent bond.

In one embodiment, X is {[N⁺(R¹) (R²) (R³) (R⁴)] [N⁺ (R⁵) (R⁶) (R⁷) (R⁸)]} wherein R¹, R², R³, R⁵, R⁶, and R⁷ are independently H, methyl, ethyl or propyl and R⁴ and R⁸ are joined by a covalent bond. In one embodiment, R⁴ and R⁸ is each independently an optionally substituted alkyl group. In one embodiment, the optional substituents for R⁴ and R⁸ is N⁺ (R¹⁰)₃. In another embodiment, one or more —CH₂— groups of R⁴ and R⁸ are replaced with a moiety selected from the group consisting of O, NH, S, S(O), and S(O)₂.

In one embodiment, X is {[N⁺ (R¹) (R²) (R³) (R⁴)] [N⁺ (R⁵) (R⁶) (R⁷) R⁸)]}, and is:

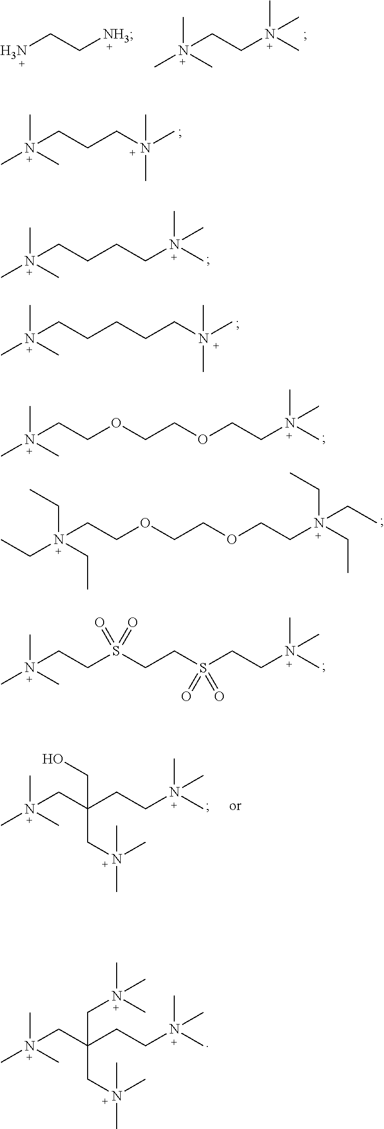

In one embodiment, X is {[N⁺ (R¹) (R²) (R³) (R⁴)] [N⁺ (R⁵) (R⁶) (R⁷) R⁸)]} wherein R¹ and R² in [N⁺ (R¹) (R²) (R³) (R⁴)]⁺ are each independently H, methyl, or ethyl and R³ and R⁴ are each independently an optionally substituted alkyl, aryl, or aralkyl group. In another embodiment, X is {[N⁺ (R¹) (R²) (R³) (R⁴)] [N⁺ (R⁵) (R⁶) (R⁷) R⁸)]} wherein R⁵ and R⁶ in [N⁺R⁵) (R⁶) (R⁷) (R⁸)] are each independently H, methyl, ethyl or propyl and R⁷ and R⁸ are each independently an optionally substituted alkyl, aryl, or aralkyl group. In one embodiment, the optional substituents for R³, R⁴, R⁷ and R⁸ are OH.

In one embodiment, [N⁺(R¹) (R²) (R³) (R⁴)]⁺ and/or [N⁺(R⁵) (R⁶) (R′) (R⁸)]⁺ in {[N⁺ (R¹) (R²) (R³) (R⁴)] [N⁺ (R⁵) (R⁶) (R⁷) R⁸)]}⁺² is independently:

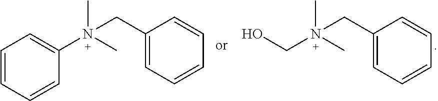

In one embodiment, X is {[N⁺ (R¹) (R²) (R³) (R⁴)] [N⁺ (R⁵) (R⁶) (R⁷) R⁸)]} wherein R¹ and R⁴ in [N⁺(R¹) (R²) (R³) (R⁴)]⁺ are each independently H, methyl, ethyl or propyl and R² and R³ together with N may form an optionally substituted cyclic structure.

In another embodiment, X is {[N⁺ (R¹) (R²) (R³) (R⁴)] [N⁺ (R⁵) (R⁶) (R⁷) R⁸)]} wherein R⁵ and R⁸ in [N⁺ (R⁵) (R⁶) (R⁷) (R⁸)]⁺are each independently H, methyl, ethyl or propyl, and R⁶ and R⁷ together with N may form an optionally substituted cyclic structure. In one embodiment, one or more —CH₂— groups in R², R³, R⁶ and R⁷ may he replaced with a moiety selected from the group consisting of O, NH, S, S(O), and S(O)₂.

In one embodiment, [N⁺(R¹) (R²) (R³) (R⁴)] and/or [N⁺(R⁵) (R⁶) (R⁷) (R⁸)] in {[N⁺ (R¹) (R²) (R³) (R⁴)] [N⁺ (R⁵) (R⁶) (R⁷) R⁸)]} is independently:

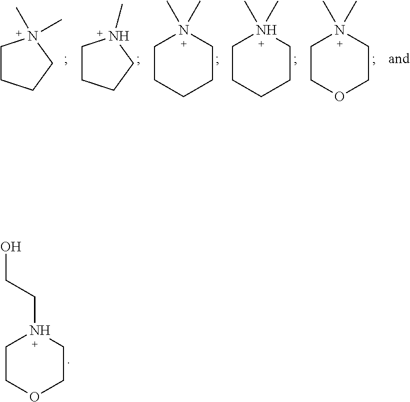

In one embodiment, X is {[N⁺ (R¹) (R²) (R³) (R⁴)] [N⁺ (R⁵) (R⁶) (R⁷) R⁸)]} wherein R⁴ and/or R⁸ is absent and R¹ and R² and/or R⁵ and R⁶ together with N forms a optionally substituted 5- or 6-membered aromatic ring, wherein up to 2 carbon atoms in the ring may be replaced with a heteroatom selected from the group consisting of O, N, and S.

In one embodiment, [N⁺(R¹) (R²) (R³) (R⁴)]⁺ and/or [N⁺(R⁵) (R⁶) (R⁷) (R⁸)]⁺ in {[N⁺ (R¹) (R²) (R³) (R⁴)] [N⁺ (R⁵) (R⁶) (R⁷) R⁸)]}⁺² is independently:

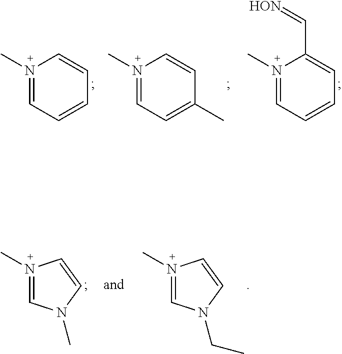

In one embodiment, X is {[N⁺ (R¹) (R²) (R³) (R⁴)] [N⁺ (R⁵) (R⁶) (R⁷) R⁸)]} wherein R¹, R², R³, R⁴, R⁵, R⁶, R⁷ and R⁸ are each H.

In one embodiment, the compound of Formula (I) is ammonium tetrathiomolybdate

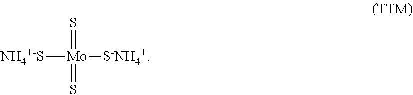

In another embodiment, the compound of Formula (I) is ammonium tetrathiotungstate

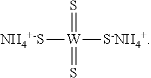

In one embodiment, X is (2Li)⁺², (2K)⁺², (2Na)⁺², Mg⁺², Ca⁺² or Zn⁺².

In one embodiment, the compound of Formula (I) is Zn(OAc)₂.

In one embodiment, the copper chelator compound is a compound of Formula (II):

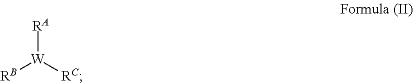

or a deprotonated anion, isomer, deuterated analog, solvate, hydrate, hydrolysis product or a pharmaceutically acceptable salt thereof, wherein:
W is N, O, or S;
R^{A}, R^{B}, and R^{C} are each independently H, alkyl, aryl, heteroaryl, cycloalkyl, heterocycoalkyl, aralkyl, alkylaralkyl, heteroaralkyl, cycloalkylalkyl, or heterocycloalkylalkyl, provided that when W is O or S, R^{C} is absent;
wherein when R^{A}, R^{B}, and/or R^{C} are alkyl, one or more carbon atoms of alkyl may be replaced with O, NH, NR¹¹, S, S(O), and S(O)₂, provided that no two adjacent carbon atoms are replaced with heteroatoms, wherein R¹¹ is each independently alkyl, -alkyl-COOH, —OC(O)alkyl, aryl, heteroaryl, cycloalkyl, heterocycoalkyl, aralkyl, alkylaralkyl, heteroaralkyl, cycloalkylalkyl, or heterocycloalkylalkyl;
wherein R^{A} and R^{B} together with W may form an optionally substituted cyclic structure comprising 5 to 30 atoms in the ring, wherein one or more carbon atoms in the ring may be replaced with a heteroatom selected from the group consisting of O, NH, NR¹¹, S, S(O), and S(O)₂, provided that no two adjacent carbon atoms are replaced with heteroatoms;
wherein two R¹¹ may join to form an optionally substituted cyclic structure comprising 5 to 30 atoms in the ring, wherein one or more carbon atoms in the ring may be replaced with a heteroatom selected from the group consisting of O, NH, S, S(O), and S(O)₂, provided that no two adjacent carbon atoms are replaced with heteroatoms;
wherein R^{A}, R^{B} and R^{C} are optionally substituted with one or more halogen, —OH, —SH, —COOH, oxo, alkyl, alkenyl, alkynyl, NH₂, NHR⁹, N(R⁹)₂, —C═N(OH) or OPO₃H₂, wherein R⁹ is each independently alkyl, —C(═O)O-alkyl, —C(═O)-alkyl, aryl, heteroaryl, aralkyl, or heteroarylalkyl;
wherein the deprotonated anion of Formula (II) indicates that one or more H⁺ from OH or SH has been removed to provide O⁻ or S⁻, respectively.

In one embodiment, R^{A}, R^{B}, and R^{C} are each independently H or optionally substituted alkyl, heteroaryl, aryl, aralkyl, or heteroarylalkyl. In another embodiment, R^{A}, R^{B}, and R^{C} are each independently H or optionally substituted pyridine, —C₁-C₃ alkyl-pyridine, or —C₁-C₃ alkyl-phenyl.

In one embodiment, W is N and R^{A}, R^{B}, and R^{C} are each independently H or optionally substituted alkyl, heteroaryl, aryl, aralkyl, or heteroarylalkyl. In another embodiment, W is N and R^{A}, R^{B}, and R^{C} are each independently H or optionally substituted pyridine, —C₁-C₃ alkyl-pyridine, or —C₁-C₃ alkyl-phenyl. In one embodiment, the optional substituents for R^{A}, R^{B}, and R^{C} are each independently selected from halogen, alkyl, NH₂, NHC(O)O-alkyl, NHC(O)alkyl, N(aralkyl)₂, N(heteroaralkyl)₂ or N(aralkyl)(heteroaralkyl).

In one embodiment, a compound of Formula (II) is a dipicolylamine or a tris(2-pyridylmethyl)amine (TPA). In another embodiment, a compound of Formula (II) is an optionally substituted dipicolylamine or TPA.

In some embodiments, dipicolylamine, shown below, can be substituted at any position of the pyridyl ring, methylene carbons, and on the sp³ nitrogen. In one embodiment, optionally substituents are selected from one or more halogen, —OH, —SH, —COOH, oxo, alkyl, alkenyl, alkynyl, NH₂, NHR⁹, N(R⁹)₂, —C═N(OH), or OPO₃H₂, wherein R⁹ is each independently alkyl, —C(═O)O-alkyl, —C(═O)-alkyl, aryl, heteroaryl, aralkyl, or heteroarylalkyl. In other embodiments, the substituents can further be substituted with the above noted substituents.

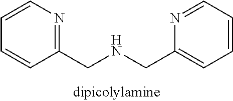 [SMILES: c1ccc(CNCc2ccccn2)nc1]

In some embodiments, TPA, shown below, can be substituted at any position of the pyridyl ring as well as on the methylene carbons. In one embodiment, optionally substituents are selected from one or more halogen, —OH, —SH, —COOH, oxo, alkyl, alkenyl, alkynyl, NH₂, NHR⁹, N(R⁹)₂, —C═N(OH), or OPO₃H₂, wherein R⁹ is each independently alkyl, —C(═O)O-alkyl, —C(═O)-alkyl, aryl, heteroaryl, aralkyl, or heteroarylalkyl. In other embodiments, the substituents can further be substituted with the above noted substituents.

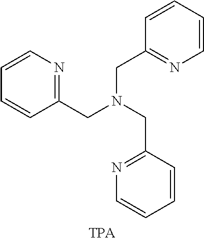 [SMILES: c1ccc(CN(Cc2ccccn2)Cc2ccccn2)nc1]

In one embodiment, a compound of Formula (II) is selected from:

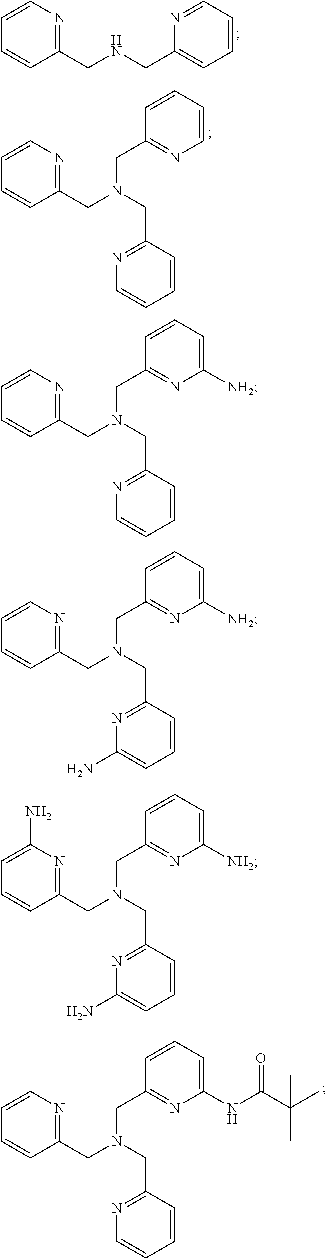

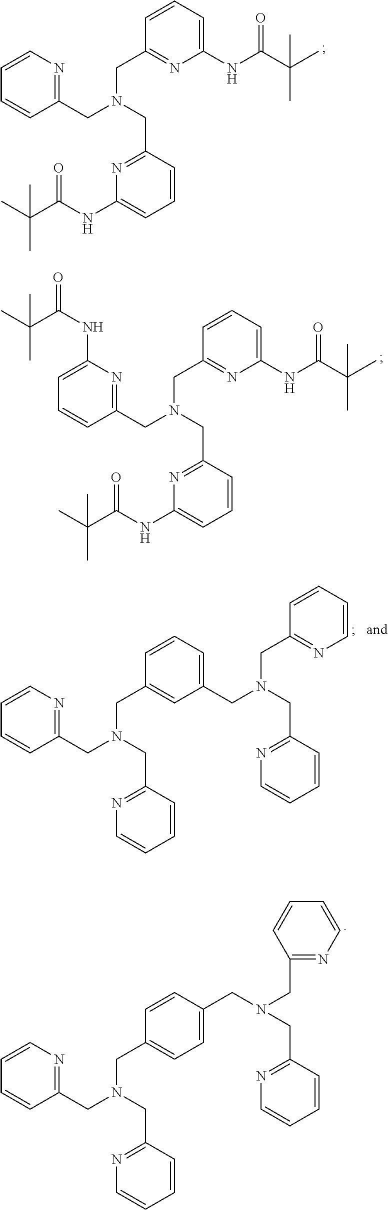

In one embodiment, R^{A}, R^{B}, and R^{C} are each independently H or an optionally substituted alkyl. In another embodiment, W is N and R^{A}, R^{B}, and R^{C} are each independently H or an optionally substituted alkyl. In one embodiment, the optional substituents for R^{A}, R^{B}, and R^{C} are each independently halogen, alkyl, NH₂, NHC(O)O-alkyl, NHC(O)alkyl, N(alkyl)₂, N(aralkyl)₂, N(heteroaralkyl)₂, N(aralkyl)(heteroaralkyl), or —COOH. In another embodiment, the optional substituents for R^{A}, R^{B}, and R^{C} are each independently halogen, oxo, alkyl, NH₂, —OH, —SR, or —COOH.

In one embodiment, a compound of Formula (II) is ethylenediaminetetraaceticacid (EDTA):

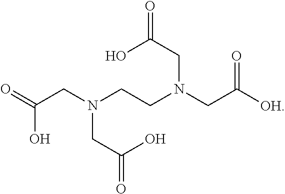 [SMILES: O=C(O)CN(CCN(CC(=O)O)CC(=O)O)CC(=O)O]

In another embodiment, R^{A}, R^{B}, and R^{C} are each independently H or an optionally substituted alkyl where one or more carbon atoms may be replaced with O, NH, NR¹¹, S, S(O), and S(O)₂, provided that no two adjacent carbon atoms are replaced with heteroatoms, wherein R¹¹ is each independently alkyl, -alkyl—COOH, —OC(O)alkyl, aryl, heteroaryl, cycloalkyl, heterocycoalkyl, aralkyl, alkylaralkyl, heteroaralkyl, cycloalkylalkyl, or heterocycloalkylalkyl. In one embodiment, the optional substituents for R^{A}, R^{B}, and R^{C} are each independently halogen, alkyl, NH₂, NHC(O)O-alkyl, NHC(O)alkyl, N(alkyl)₂, N(aralkyl)₂, N(heteroaralkyl)₂, N(aralkyl)(heteroaralkyl), or —COOH.

In one embodiment, a compound of Formula (II) is an optionally substituted acyclic polyether, acyclic crown ether, acyclic polyamine, acyclic polythioether, where one or more carbon atoms may be replaced with O, NH, NR¹¹, S, S(O), and S(O)₂, provided that no two adjacent carbon atoms are replaced with heteroatoms

In one embodiment, R^{A}, R^{B}, and R^{C} are each independently H or an optionally substituted alkyl where one or more carbon atoms are replaced with NH, provided that no two adjacent carbon atoms are replaced.

In one embodiment, a compound of Formula (II) is a polyamine. Non-limited examples of polyamine include triethylenetetramine, ethylenediamine, and diethylenetriamine.

In one embodiment, a compound of Formula (II) is:

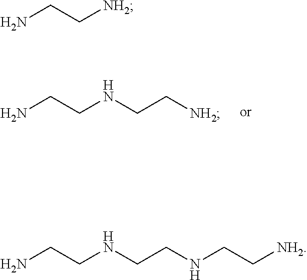

In one embodiment, a compound of Formula (II) is D-penicillamine:

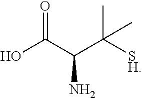

In one embodiment, a compound of Formula (II) is glutathione:

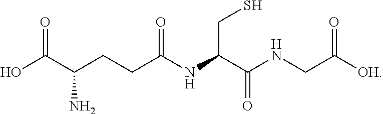 [SMILES: N[C@@H](CCC(=O)N[C@@H](CS)C(=O)NCC(=O)O)C(=O)O]

In one embodiment, W is O or S. In another embodiment, W is O or S and R^{A} and R^{B} are each independently H or an optionally substituted alkyl. In one embodiment, the optional substituents for R^{A} and R^{B} are each independently halogen, alkyl, NH₂, —OH, —SH, or —COOH.

In e embodiment, a compound of Formula (II) is dimercaprol:

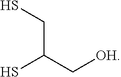

In one embodiment, R^{A} and R^{B} together with W forms an optionally substituted cyclic structure comprising 5 to 30 atoms in the ring, wherein one or more carbon atoms in the ring may be replaced with a heteroatom selected from the group consisting of O, NH, NR¹¹, S, S(O), and S(O)₂, provided that no two adjacent carbon atoms are replaced with heteroatoms, wherein R¹¹ is each independently alkyl, -alkyl-COOH, —OC(O)alkyl, aryl, heteroaryl, cycloalkyl, heterocycoalkyl, aralkyl, alkylaralkyl, heteroaralkyl, cycloalkylalkyl, or heterocycloalkylalkyl. In one embodiment, W is O or N and R^{A} and R^{B} together with W forms an optionally substituted cyclic structure comprising 5 to 30 atoms in the ring, wherein one or more carbon atoms in the ring may be replaced with a heteroatom selected from the group consisting of O, NH, NR¹¹, S, S(O), and S(O)₂, provided that no two adjacent carbon atoms are replaced with heteroatoms, wherein R¹¹ is each independently alkyl, -alkyl-COOH, —OC(O)alkyl, aryl, heteroaryl, cycloalkyl, heterocycoalkyl, aralkyl, alkylaralkyl, heteroaralkyl, cycloalkylalkyl, or heterocycloalkylalkyl. In another embodiment, R^{A} and R^{B} are both alkyl and together with W form an optionally substituted cyclic structure comprising 5 to 30 atoms in the ring.

In one embodiment, a compound of Formula (II) is a crown ether, aza-crown ether, cyclam, or cyclen. In another embodiment, a compound of Formula (II) is an optionally substituted crown ether, aza-crown ether, cyclam, or cyclen.

In one embodiment, a compound of Formula (II) is:

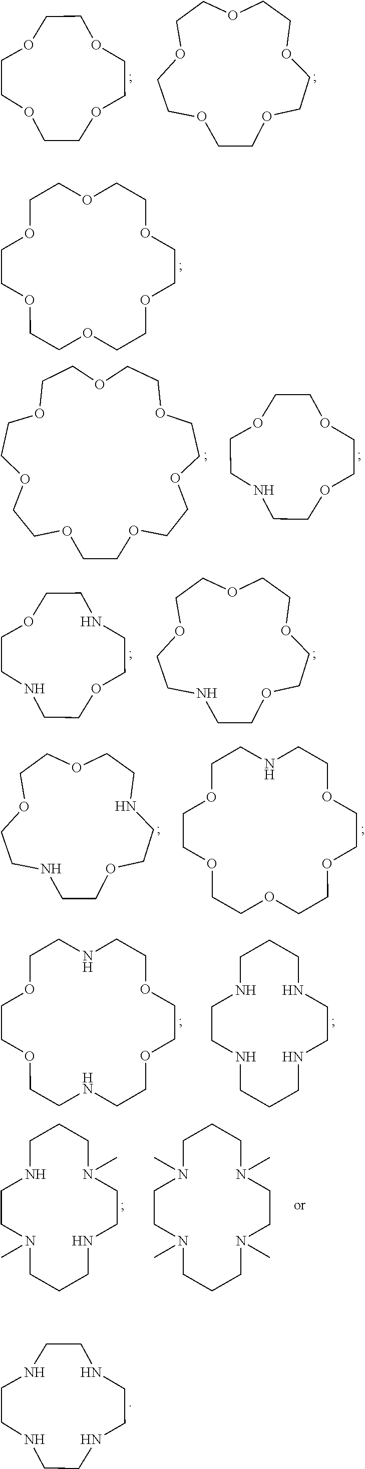

In one embodiment, two R¹¹ joins to form an optionally substituted cyclic structure comprising 5 to 30 atoms in the ring, wherein one or more carbon atoms in the ring may be replaced with a heteroatom selected from the group consisting of O, NH, S, S(O), and S(O)₂, provided that no two adjacent carbon atoms are replaced with heteroatoms.

In one embodiment, a compound of Formula (II) is a cryptand. In another embodiment, a compound of Formula (II) is an optionally substituted cryptand.

In one embodiment, a compound of Formula (II) is:

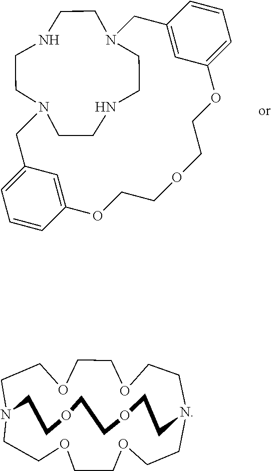

In one embodiment of Formula (II), W is N, R^{A} is H, and R^{C} is alkyl, aralkyl, or heteroarylalkyl, wherein R^{C} is substituted with at least one —COOH. In another embodiment, W is N, R^{A} is H, and R^{C} is alkyl, aralkyl, or heteroarylalkyl, wherein R^{C} is substituted with at least one —COOH, where the carboxylic acid is deprotonated to provide a —COO⁻ moiety to form a deprotonated anion. In another embodiment, one or more of the deprotonated anion can chelate a metal, for example, Mo(II), Mo(IV), and Mo(VI).

In one embodiment, Formula (II) comprises one or more functional group selected from: —SH, —OH, —COOH, or OPO(OH)₂, where one or more H in the group listed are deprotonated to provide a deprotonated anion of Formula (II). Deprotonated anion of Formula (II), in some embodiments, can chelate to a metal species, for example, Mo(II), Mo(IV), and Mo(VI).

In some embodiments, a deprotonated anion of Formula (II) can chelate to a metal to form a complex such as molybdenum amino acid chelate, e.g., molybdenum glycinate, or molybdenum cofactor.

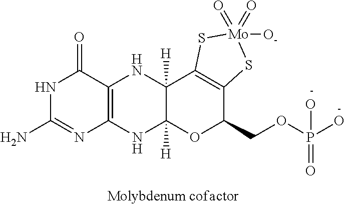

In yet another embodiment, the copper chelator is an amino acid or peptide complex of a metal species. For example, Mo(ii), Mo(iv), and Mo(vi) can each complex with an amino acid or peptide to form an Mo chelate. The peptide in one embodiment, is from about two amino acids to about nine amino acids in length and can include both natural and non-natural amino acids.

The term “amino acid” refers to both natural (genetically encoded) and non-natural (non-genetically encoded) amino acids, and moieties thereof. Of the twenty natural amino acids, 19 have the general structure:

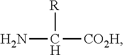

where R is the amino acid side chain. The 20^{th} amino acid, proline, is also within the scope of the present invention, and has the following structure:

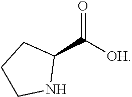

Of the twenty natural amino acids, all but glycine is chiral, and both the D- and L-amino acid isomers, as well as mixtures thereof, are amenable for use with the present invention. It is also noted that an amino acid moiety is encompassed by the term “amino acid.” For example, the amino acid moieties:

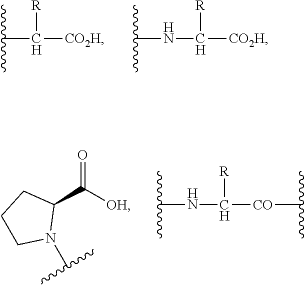

are encompassed by the term “amino acid.”

In one embodiment, the Mo amino acid chelate includes one or more five-membered rings formed by a reaction between the amino acid (or peptide) and the molybdate. See for example, U.S. Pat. Nos. 5,516,925 and 6,716,814, both of which are incorporated by reference herein in their entireties for all purposes.

The molybdate amino acid chelate in one embodiment includes a homogeneous population of amino acids. In another embodiment, the molybdate amino acid/peptide chelate includes a heterogeneous population of amino acids.

The molybdate peptide chelate in one embodiment includes a homogeneous population of peptides. In another embodiment, the molybdate amino acid/peptide chelate includes a heterogeneous population of peptides.

The molybdate amino acid/peptide chelate can include molybdate at the following oxidation states: Mo(ii), Mo(iv), and Mo(vi)].

As provided herein, the composition provided herein includes a pharmaceutically acceptable carrier, diluent or excipient. The pharmaceutically acceptable carrier, diluent or excipient in one embodiment is a solubilizing agent, an antioxidant, a stabilizing agent or a combination thereof.

Compositions provided herein can be formulated as dry powders, solutions or suspensions.

The “pharmaceutically acceptable carrier, diluent or excipient” includes any and all solvents, diluents, or other liquid vehicle, dispersion or suspension aids, surface active agents, isotonic agents, thickening or emulsifying agents, preservatives, solid binders, lubricants and the like, as suited to the inhalation dosage form provided herein. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. “Pharmaceutically acceptable excipient or carrier” also relates to an excipient or carrier that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

In one embodiment, the composition provided herein comprises a modified release or controlled release component. In a further embodiment, the composition is an oral dosage form.

For example, one or more of the following components can be utilizes as the modified release or controlled release component: cellulose acetate phthalate, cellulose acetate trimaletate, hydroxy propyl methylcellulose phthalate, polyvinyl acetate phthalate, ammonio methacrylate copolymers such as those sold under the Trademark Eudragit® RS and RL, poly acrylic acid and poly acrylate and methacrylate copolymers such as those sold under the Trade Mark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium. carmellose, sodium carboxymethyl starch, poly vinyl alcohol, hydroxyethyl cellulose, methyl cellulose, gelatin, starch, and cellulose based cross-linked polymers in which the degree of crosslining is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, arninoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (MW^{˜}5 k-5,000 k), polyvinylpyrrolidone (MW^{˜}10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (MW^{˜}30 k-300 k), polysaccharides such as agar, acacia, karaya, tragacanth, algins and guar, polyacrylamides, Polyox® polyethylene oxides (MW^{˜}100 k-5,000 k), AquaKeep® acrylate polymers, diesters of polyglucan, crosslinked polyvinyl alcohol and poly N-vinyl-2-pyrrolidone, sodium starch glucolate (e.g., Explotab®, Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyoxe®, Union Carbide), methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic, acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

In one embodiment, the modified release component is a biodegradable polymer. In a further embodiment, the biodegradable polymer comprises a monosaccharide, disaccharide, polysaccharide, peptide, protein, or protein domain. The protein in one embodiment comprises only natural amino acids. However, non-natural amino acids, or protein domains thereof, can also be used as monomer components of the polymer.

Other modified release agents amenable for use as a modified release component include but are not limited to, hyaluronic acid and polymers thereof, polyamino acids (natural and non-natural amino acids, including peptides and proteins), poly(lactic-co-glycolic acid), polycaprolactone, polyglycolide, polylactic acid, polyhydroxybutyrate or a combination thereof.

In one embodiment, a polymer comprised of one or more of the following monomers is employed as a monomer component of a modified release polymer: lactic acid, glycolic acid, acrylic acid, 1-hydroxyethyl methacrylate, ethyl methacrylate, 2-hydroxyethyl methacrylate (HEMA), propylene glycol methacrylate, acrylamide, N-vinylpyrrolidone (NVP), methyl methacrylate, glycidyl methacrylate, glycerol methacrylate (GMA), glycol methacrylate, ethylene glycol, fumaric acid, a derivatized version thereof, or a combination thereof.

As will be appreciated by the person of ordinary skill in the art, excipients such as plasticisers, lubricants, solvents and the like may also be added. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl di-2-ethylhexyl adipate, di-2-ethylhexyl sebacate, di-2-ethylhexyl azelate, dibutyl sebacate.

The modified release component can be present in a layer of an oral dosage form, e.g., a coating, or as a matrix material. Matrix materials that are amenable for use herein include hydrophilic polymers, hydrophobic polymers and mixtures thereof which are capable of modifying the release of a copper chelator compound dispersed therein in vitro or in vivo. Modified release matrix materials suitable for the practice of the present invention include but are not limited to microcrytalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpynolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

Compositions provided herein in one embodiment include an antioxidant, such as acetone sodium bisulfate, ascorbic acid; preservatives, such as ammonia, benzalkonium chloride, cetylpyridinium chloride, chlorobutanol, glycerin, methylparaben, propylparaben, propylene glycol, sodium metabisulfite, sodium sulfite; wetting, emulsification, dispersion, solubilization agents, suspension aids and valve lubricants such as benzalkonium chloride, lecithin (soya), magnesium stearate, oleic acid, polysorbate 80, polyvinylpyrrolidone K25, sorbitan trioleate (Span 85), Thymol, Pluronic® F-77, Pluronic® F-68, Pluronic® L-92, Pluronic® L-121, polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether, methyl polyethylene glycol (f-mPEG), oligolactic acid (OLA), hydrophobic counterions (e.g., lauric acid, lauroyl sarcosine and lauroyl lactylate) and hydrophilic counterions (e.g., functionalized polyethers), acetylated cyclodextrins; flavorings, such as citric acid (anhydrous), menthol, saccharin, saccharin sodium dehydrate, sodium citrate; chelating agents, such as edetate sodium/edetate disodium, sodium citrate; cosolvents, such as ethanol, dehydrated alcohol, alcohol, glycerin, propylene glycol, water; humectants, such as glycerin; tonicity agents, such as glycerin, sodium chloride, sodium sulfate (anhydrous); buffering agents, such as glycine, lysine monohydrate, sodium citrate, tromethamine; drug stabilizers, such as glycine, lysine monohydrate; pH adjustors, such as hydrochloric acid, nitric acid, sodium bisulfate, sodium hydroxide, sulfuric acid.

In another embodiment, the composition comprises an effective amount of a copper chelator compound, a hydrolysis product thereof, or a pharmaceutically acceptable salt thereof and a solubilization agent. In a further embodiment, the solubilization agent is Pluronic® F-77, Pluronic® F-68, Pluronic® L-92, Pluronic® L-121, polyethylene glycol, diethylene glycol monoethyl ether, polyoxyethylene sorbitan monolaurate, polyoxyethylene sorbitan monooleate, propoxylated polyethylene glycol, polyoxyethylene lauryl ether, methyl polyethylene glycol (f-mPEG), oligolactic acid (OLA), hydrophobic counterions, hydrophilic counterions, acetylated cyclodextrins, or combinations thereof.

In one embodiment, the solubilization agent is an organic acid. In a further embodiment, the organic acid is acetic acid, ascorbic acid, citric acid, lactic acid, malic acid, succinic acid, or a combination thereof.

In yet another embodiment, the composition comprises an effective amount of a copper chelator compound and a suspension aid. In another embodiment, the suspension aid is oleic acid, polysorbate 80, polyvinylpyrrolidone K25, or combinations thereof.

In one embodiment, the copper chelators composition comprises a CAR peptide, e.g., the peptide of SEQ ID NO: 1 (CARSKNKDC) or a variant thereof, e.g., the peptide of, SEQ ID NO: 2 (CARSKNK) or SEQ ID NO: 3 (CAQSNNKDC). CARSKNKDC (SEQ ID NO: 1) (CAR) peptide has been previously been shown to target wound healing (Järvinen and Ruoslahti (2007). The American Journal of Pathology, 171, pp. 702-711, incorporated by reference herein in its entirety for all purposes). The CAR peptide can be linear or cyclic. Additionally, the CAR peptide can be complexed to the copper chelator compound or present separately in the composition. In one embodiment, the CAR peptide is conjugated to a lipid component, for example one of the lipid components described herein. The targeting peptide, e.g., the CAR peptide in one embodiment is conjugated to decorin, a small chondroitin/dermatan sulfate proteoglycan, e.g., as described previously by Järvinen and Ruoslahti (2010). PNAS USA 107, pp. 21671-21676, incorporated by reference herein in its entirety for all purposes.

In one aspect of the invention, a composition is provided comprising a copper chelator, isomer, solvate, hydrate, hydrolysis product or pharmaceutically acceptable salt thereof, complexed to or encapsulated by a lipid component. A copper chelator is “complexed” to a lipid or a lipid component and describes any composition, solution or suspension where at least about 1% by weight of the copper chelator is associated (e.g., encapsulated or bound) with the lipid either as part of a complex, for example, as part of a microparticle, nanoparticle, micelle or liposome. The complex, in one embodiment, is formed by one or more electrostatic interactions, hydrophobic interactions, hydrogen bonds or by the encapsulation of the copper chelator by the lipid, e.g., in a micelle or liposome. For example, the lipid-complexed composition, in one embodiment, comprises liposomes, and the copper chelator may be in the aqueous phase (encapsulated by the liposome), the hydrophobic bilayer phase, at the interfacial headgroup region of the liposomal bilayer or a combination thereof. In one embodiment, prior to administration of the composition to a patient in need thereof, at least about 5%, at least about 10%, at least about 20%, at least about 25%, at least about 50%, at least about 75%, at least about 80%, at least about 85%, at least about 90% or at least about 95% of the copper chelator in the composition is lipid complexed. Association, in one embodiment, is measured by separation through a filter where lipid and lipid-associated drug is retained (i.e., in the retentate and free drug is in the filtrate.

The lipid component can comprise a homogeneous population of lipid or a heterogeneous population of lipid. That is, different lipids can be employed in the same composition, if desired. The lipid component is complexed to a copper chelator, e.g., one of the copper chelators described herein, or an isomer, solvate, hydrate, hydrolysis product or pharmaceutically acceptable salt thereof. The complex, in one embodiment, is a microparticle, nanoparticle, micelle, liposome, or a combination thereof. In a further embodiment, the composition comprises a cationic lipid, or different cationic lipids.

In one embodiment, the lipid complex is a liposome or liposomes, and the copper chelator is associated with the liposome surface, or present in the aqueous interior of the liposome (or liposomes). Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

The liposome in one embodiment is an immunoliposome. For example, the lipid component (or portion thereof) of the liposome in one embodiment is conjugated to an antibody or antigen binding portion thereof. Conjugation in one embodiment is through a biotin-avidin linkage. In one embodiment, the antibody is an anti-VEGF antibody. In a further embodiment, the anti-VEGF antibody is conjugated to biotin (see, e.g., product ab83143 from abcam (Cambridge, Mass.).

As provided above, in one embodiment, the lipid component or portion thereof of the liposome is conjugated to the CAR peptide or derivative thereof (e.g., the peptide of SEQ ID NO: 1, SEQ ID NO: 2 or SEQ ID NO: 3). Conjugation in one embodiment is to a phosphatidylethanolamine (e.g., PE MCC (CAS No. 384847-49-8), succinyl PE (CAS No. 111613-33-3) or caproylamine PE (CAS No. 115288-21-6)).

In one embodiment, when formulated together, the copper chelator and lipid component form lipid particles (e.g., microparticles or nanoparticles). In one embodiment, the lipid component is a cationic lipid, a PEGylated lipid, a surfactant or a block copolymer. In a further embodiment, the mean diameter of the lipid particles is from about 20 nm to about 2 μm, for example about 50 nm to about 1 μm, about 200 μnm to about 1 μm, about 100 nm to about 800 nm, about 100 nm to about 600 nm or about 100 nm to about 500 nm.

As provided above, in one embodiment, a cationic lipid is provided in the composition described herein together with a copper chelator. The cationic lipid, in one embodiment, includes ammonium salts of fatty acids, phospholipids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammoniu-m chloride (DOTMA), dioleylphosphatidylethanolamine (DOPE) and 1,2-bis(oleoyloxy)-3-(trimethylammonio) propane (DOTAP).

The lipid component of the present invention, in one embodiment, is a PEGylated lipid. For example, where a cationic lipid is employed, it can be derivatized with a PEG molecule to form a PEGylated lipid. The PEGylated lipid, in one embodiment, comprises PEG400-PEG5000. For example, the PEGylated lipid can comprise PEG400, PEG-500, PEG1000, PEG2000, PEG3000, PEG4000, or PEG5000. In a further embodiment the lipid component of the PEGylated lipid comprises cholesterol, dimyristoyl phosphatidylethanolamine (DMPE), dipalmitoyl phosphoethanolamine (DPPE), distearoylphosphatidylethanolamine (DSPE), dimyristoylglycerol glycerol (DMG), diphosphatidylglycerol (DPG) or disteraroylglycerol (DSG). In even a further embodiment, the PEGylated lipid is cholesterol-PEG2000 or DSPE-PEG2000.

Depending on its molecular weight (MW), PEG is also referred to in the art as polyethylene oxide (PEO) or polyoxyethylene (POE). The PEGylated lipid can include a branched or unbranched PEG molecule, and is not limited by a particular PEG MW. For example, the PEGylated lipid, in one embodiment, comprises a PEG molecule having a molecular weight of 300 g/mol, 400 g/mol, 500 g/mol, 1000 g/mol, 1500 g/mol, 2000 g/mol, 2500 g/mol, 3000 g/mol, 3500 g/mol, 4000 g/mol, 4500 g/mol, 5000 g/mol or 10,000 g/mol. In one embodiment, the PEG has a MW of 1000 g/mil or 2000 g/mol.

The lipid component, in one embodiment, comprises a non-phospholipid such as a ceramide. In a further embodiment, the ceramide is present in liposomes.

The lipid component can have a net-charge (e.g., cationic or anionic), or can be net-neutral. The lipids used in the lipid component (PEGylated or non-PEGylated) of the present invention can be synthetic, semi-synthetic or naturally-occurring lipid, including a phospholipid, a sphingolipid, a glycolipid, a ceramide, a tocopherol, a sterol, a fatty acid, or a glycoprotein such as albumin.

The lipid component, for example, comprises a negatively charged lipid, for example, a negatively charged phospholipid. In one embodiment, the negatively charged lipid comprises dihexadecylphosphate (DHP). In one embodiment, the negatively charged phospholipid is a phosphatidylserine (PS) and/or phosphatidylglycerol (PG). The phosatidylserine and/or phosphatidylglycerol can be any phosphatidylserine known to those of ordinary skill in the art. For example, the PS in one embodiment is egg phosphatidylserine (EPS), dilauroyl-phosphoserine (DLPS), dimyristoylphosphoserine (DMPS), dioleoyl-phosphoserine (DOPS), di palmitoyl-phosphoserine (DPPS), distearoyl-phosphoserine (DSPS) or a combination thereof. The PG, in one embodiment, is egg phosphatidylglycerol (EPG), dipalmitoylphosphatidylglycerol (DPPG), dioleoyl-glycero-phosphatidylglycerol (DOPG), dimyristoylphosphatidylglycerol (DMPG), distearoylphosphatidylglycerol (DSPC), palmitoyl-oleoyl-phosphatidylglycerol (POPE), or a combination thereof. Combinations of negatively charged lipids can also be employed.

In one embodiment, the lipid component comprises one or more negatively charged lipids and one or more net neutral lipids, for example, a net neutral phospholipid, cholesterol or a combination thereof. The net neutral phospholipid in one embodiment is a phosphatidylcholine. In a further embodiment, the phosphatidylcholine is egg phosphatidylcholine, dipalmitoylphosphatidylcholine (DPPC), distearoylphosphatidylcholine (DSPC), 1,2-Oleoyl-sn-glycero-3-phosphocholine (DOPC), dimyristoylphosphatidylcholine (DMPC), lysolecithin or a combination thereof.

In one embodiment, the lipid comprises a sterol. In a further embodiment, the sterol is cholesterol. In another embodiment, the lipid comprises a phospholipid, for example a negatively charged lipid, a net neutral lipid and a sterol. Phospholipids include, but are not limited to phosphatidylcholine (PC), phosphatidylglycerol (PG), phosphatidylinositol (PI), phosphatidylserine (PS), phosphatidylethanolamine (PE), and phosphatidic acid (PA). In one embodiment, the phospholipid is an egg phospholipid, a soya phospholipid or a hydrogenated egg and soya phospholipid.

In one embodiment, the lipid component comprises a PEGylated lipid and the PEGylated lipid comprises a phospholipid. In a further embodiment, the phospholipid comprises ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, in one embodiment, the PEGylated lipid of the composition provided herein comprises distearoylphosphoethanolamine (DSPE), dipalmitoylphosphatidylcholine (DPPC), dioleoylphosphatidylcholine (DOPC) dimyristoyl phosphatidylethanolamine (DMPE), dipalmitoylphosphoethanolamine (DPPE), distearoylphosphatidylethanolamine (DSPE), dimyristoylglycerol (DMG), diphosphatidylglycerol (DPG) or disteraroylglycerol (DSG).

Other examples of lipids for use in the compositions provided herein (PEGylated or non-PEGylated) include dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG) dioleylphosphatidylethanolamine (DOPE), and mixed phospholipids such as palmitoylstearoylphosphatidylcholine (PSPC) and palmitoylstearoylphosphatidylglycerol (PSPG), triacylglycerol, diacylglycerol, ceramide, sphingosine, sphingomyelin and single acylated phospholipids such as mono-oleoyl-phosphatidylethanolamine (MOPE). In another embodiment lipid component of the composition comprises an ammonium salt of a fatty acid, a phospholipid, a glyceride, a phospholipid and glyceride, a sterol (e.g., cholesterol), phosphatidylglycerol (PG), phosphatidic acid (PA), a phosphatidylcholine (PC), a phosphatidylinositol (PT), a phosphatidylserine (PS), or a combination thereof. The fatty acid, in one embodiment, comprises fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9(Z)-octadecenyloxy)-prop-1-yl-N,N,N-trimethylammonium chloride (DOTMA) and 1,2-bis(pleoyloxy)-3-(trimethylammonio)propane (DOTAP). Examples of sterols for use in the lipid particle compositions provided herein include cholesterol and ergosterol. Examples of PGs, PAs, PIs, PCs and PSs for use in the compositions provided herein include DMPG, DPPG, DSPG, DMPA, DPPA, DSPA, DMPI, DPPI, DSPI, DMPS, DPPS and DSPS, DSPC, DPPG, DMPC, DOPC, egg PC and soya PC.

In one embodiment, the lipid component is a PEGylated lipid and is cholesterol-PEG2000, DSPE-PEG1000 or DSG-PEG2000.

In yet another embodiment, two or more copper chelators, a lipid component (e.g., a cationic lipid, PEGylated lipid, a phospholipid, a sterol, or combination thereof) and a hydrophobic additive are provided in a composition, for example, a composition comprising microparticles or nanoparticles of a copper chelator complexed to the lipid component.

In one lipid particle embodiment, the copper chelator is present in the composition at 5 mol %-99 mol %. In a further embodiment, the copper chelator is present in the composition at 40 mol % 95 mol %. In a further embodiment, the copper chelator is present in the composition at 40 mol %-60 mol %. In one embodiment, the copper chelator present in the composition at about 40 mol % or about 45 mol %.

The lipid component, e.g., a PEGylated lipid, in one embodiment, is present in the composition at 10 mol %-30 mol %, for example, 10 mol %-20 mol % or 15 mol %-25 mol %. In even a further embodiment, the lipid (e.g., cationic lipid) is present in the composition at about 10 mol % or 20 mol %.

In some embodiments, the compositions, systems and methods provided herein comprise a lipid complexed (e.g., liposomal encapsulated) copper chelator compound. The lipids used in the pharmaceutical compositions of the present invention as provided throughout can be synthetic, semi-synthetic or naturally-occurring lipids, including phospholipids, tocopherols, sterols, fatty acids, net-neutral lipids, negatively-charged lipids and cationic lipids.

In one embodiment, at least one phospholipid is present in the composition. In a further embodiment, the composition comprises liposomes or lipid particles comprising a lipid complexed copper chelator. In one embodiment, the phospholipid is: phosphatidylcholine (EPC), phosphatidylglycerol (PG), phosphatidylinositol (PI), phosphatidylserine (PS), phosphatidylethanolamine (PE), phosphatidic acid (PA); the soya counterparts, soy phosphatidylcholine (SPC); SPG, SPS, SPI, SPE, and SPA; the hydrogenated egg and soya counterparts (e.g., HEPC, HSPC), a phospholipid made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The carbon chains on these fatty acids can be saturated or unsaturated, and the phospholipid may be made up of fatty acids of different chain lengths and different degrees of unsaturation.

In one embodiment, the composition includes dipalmitoylphosphatidylcholine (DPPC), a major constituent of naturally-occurring lung surfactant. In one embodiment, the lipid component of the composition comprises DPPC and cholesterol, or consists essentially of DPPC and cholesterol, or consists of DPPC and cholesterol. In a further embodiment, the DPPC and cholesterol have a mole ratio in the range of from about 19:1 to about 1:1, or about 9:1 to about 1:1, or about 4:1 to about 1:1, or about 2:1 to about 1:1, or about 1.86:1 to about 1:1. In even a further embodiment, the DPPC and cholesterol have a mole ratio of about 2:1 or about 1:1.

Without wishing to be bound by theory, phosphatidylcholines, such as DPPC, aid in the uptake of the copper chelator by the cells in the lung (e.g., the alveolar macrophages) and helps to maintain the copper chelator compound in the lung. The negatively charged lipids such as the PGs, PAs, PSs and PIs, in addition to reducing particle aggregation, are thought to play a role in the sustained activity characteristics of the inhalation formulation as well as in the transport of the formulation across the lung (transcytosis) for systemic uptake. The sterol compounds, without wishing to be bound by theory, are thought to affect the release characteristics of the formulation.

Other examples of lipids for use with the lipid complexed (e.g., liposomal, micelle, lipid particle) compositions described herein include but are not limited to, dimyristoylphosphatidycholine (DMPC), dimyristoylphosphatidylglycerol (DMPG), dipalmitoylphosphatidcholine (DPPC), dipalmitoylphosphatidlglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE). The lipid component (e.g., comprising one or more lipids), as described above, can be PEGylated.

In one embodiment, the lipid component of the liposomal or lipid particle composition comprises a sterol. In a further embodiment, the lipid component of the liposomal composition comprises a sterol and a phospholipid, or consists essentially of a sterol and a phospholipid, or consists of a sterol and a phospholipid. Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates.

A variety of sterols and their water soluble derivatives such as cholesterol hemisuccinate have been used to form liposomes; see, e.g., U.S. Pat. No. 4,721,612, incorporated by reference herein in its entirety. PCT Publication No. WO 85/00968, incorporated by reference herein in its entirety, describes a method for reducing the toxicity of drugs by encapsulating them in liposomes comprising α-tocopherol and certain derivatives thereof. Also, a variety of tocopherols and their water soluble derivatives have been used to form liposomes, see PCT Publication No. 87/02219, incorporated by reference in its entirety. The methods described in these publications are amenable for use herein. In one embodiment, at least one anionic lipid (negatively charged lipid) is provided in the liposomal compositions described herein. The negatively-charged lipids which can be used include phosphatidyl-glycerols (PGs), phosphatidic acids (PAs), phosphatidylinositols (Pis) and the phosphatidyl serines (PSs). Examples are provided above and include DMPG, DPPG, DSPG, DMPA, DPPA, DSPA, DMPI, DPPI, DSPI, DMPS, DPPS and DSPS.

Liposomes can be produced by a variety of methods and the present invention is not limited to a particular type of liposomal manufacturing method. In one embodiment, one or more of the methods described in U.S. Patent Application Publication No. 2008/0089927 or WO 2013/177226 are used herein to produce the copper chelator encapsulated lipid compositions (liposomal dispersion). The disclosures of U.S. Patent Application Publication No. 2008/0089927 and PCT publication no. 2013/177226 are incorporated by reference herein in their entireties for all purposes.

In one embodiment, the liposomal composition is formed by dissolving one or more lipids in an organic solvent forming a lipid solution, and a copper chelator coacervate forms from mixing an aqueous solution of the copper chelator with the lipid solution. In a further embodiment, the organic solvent is ethanol. In even a further embodiment, the one or more lipids comprise a phospholipid and a sterol. The phospholipid, in one embodiment is net neutral or net cationic.

In one embodiment, liposomes are produces by sonication, extrusion, homogenization, swelling, electroformation, inverted emulsion or a reverse evaporation method. Bangham's procedure (J. Mol. Biol. (1965)) produces ordinary multilamellar vesicles (MLVs). Lenk et al. (U.S. Pat. Nos. 4,522,803, 5,030,453 and 5,169,637, each incorporated by reference in their entireties for all purposes), Fountain et al, (U.S. Pat. No. 4,588,578, incorporated by reference in its entirety) and Cullis et al. (U.S. Pat. No. 4,975,282, incorporated by reference in its entirety) disclose methods for producing multilamellar liposomes having substantially equal interlamellar solute distribution in each of their aqueous compartments. U.S. Pat. No. 4,235,871, incorporated by reference in its entirety, discloses preparation of oligolamellar liposomes by reverse phase evaporation. Each of the methods is amenable for use with the present invention.

Unilamellar vesicles can be produced from MLVs by a number of techniques, for example, the extrusion techniques of U.S. Pat. No. 5,008,050 and U.S. Pat. No. 5,059,421, the disclosure of each of which is incorporated by reference herein for all purposes. Sonication and homogenization cab be so used to produce smaller unilamellar liposomes from larger liposomes (see, for example, Paphadjopoulos et al. (1968); Deamer and Uster (1983); and Chapman et al. (1968), each of which is incorporated by reference in its entirety for all purposes).

The liposome preparation of Bangham et al. (J. Mol. Biol. 13, 1965, pp. 238-252, incorporated by reference in its entirety) involves suspending phospholipids in an organic solvent which is then evaporated to dryness leaving a phospholipid film on the reaction vessel. Next, an appropriate amount of aqueous phase is added, the 60 mixture is allowed to “swell,” and the resulting liposomes which consist of multilamellar vesicles (MLVs) are dispersed by mechanical means. This preparation provides the basis for the development of the small sonicated unilamellar vesicles described by Papahadjopoulos et at (Biochim. Biophys. Acta. 135, 1967, pp. 624-638, incorporated by reference in its entirety), and large unilamellar vesicles.

Techniques for producing large unilamellar vesicles (LANs), such as, reverse phase evaporation, infusion procedures, and detergent dilution, can be used to produce liposomes for use in the pharmaceutical compositions provided herein. A review of these and other methods for producing liposomes may be found in the text Liposomes, Marc Ostro, ed., Marcel Dekker, Inc., New York, 1983, Chapter 1, which is incorporated herein by reference in its entirety for all purposes. See also, Szoka, Jr. et al., (Ann. Rev. Biophys, Bioeng. 9, 1980, p. 467), which is also incorporated herein by reference in its entirety for all purposes.

Other techniques for making liposomes amenable for making the compositions described herein include those that form reverse-phase evaporation vesicles (REV), see, e.g., U.S. Pat. No. 4,235,871, incorporated by reference in its entirety. Another class of liposomes that may be used is characterized as having substantially equal lamellar solute distribution. This class of liposomes is denominated as stable plurilamellar vesicles (SPLV) as defined in U.S. Pat. No. 4,522,803, incorporated by reference in its entirety, and includes monophasic vesicles as described in U.S. Pat. No. 4,588,578, incorporated by reference in its entirety, and frozen and thawed multilamellar vesicles (FATMLV) as described above.

The composition, in one embodiment comprises lipid particles with a mean diameter that is measured by a light scattering method, of about 0.005 microns to about 3.0 microns, for example, in the range about 0.1 μm to about 1.0 μm. In one embodiment, the mean diameter of the lipid particles in the composition is about 50 nm to about 2 μm, about 50 nm to about 1.5 μm, about 50 nm to about 1 μm, 50 nm to about 900 nm, about 50 nm to about 800 nm, about 50 nm to about 700 nm, about 50 nm to about 600 nm, about 50 nm to about 500 nm. In another embodiment, the mean diameter of the lipid particles in the composition is from about 200 nm to about 1.8 μm, from about 200 nm to about 1.7 μm, from about 200 nm to about 1.6 μm, from about 200 nm to about 1.5 μm, from about 200 nm to about 1.4 μm, from about 200 nm to about 1.3 μm, from about 200 nm to about 1.2 μm or from about 200 nm to about 1.1 μm.

The lipid particles in one embodiment, comprise a liposomes. In one embodiment, the liposomes have a mean diameter that is measured by a light scattering method, of about 0.01 microns to about 3.0 microns, for example, in the range about 0.2 to about 1.0 microns. In one embodiment, the mean diameter of the liposomes in the composition is about 150 nm to about 2 μm, about 200 nm to about 1.9 μm, about 200 nm to about 1.8 μm, about 200 nm to about 1.7 μm, about 200 nm to about 1.6 μm, about 200 nm to about 1.5 μm, about 200 nm to about 1.4 μm, about 200 nm to about 1.3 μm, about 200 nm to about 1.2 μm, about 200 nm to about 1.1 μm, about 200 nm to about 1 μm, 200 nm to about 900 nm, about 200 nm to about 800 nm, about 200 nm to about 700 nm, about 200 nm to about 600 nm, about 200 nm to about 500 nm.

In order to minimize dose volume and reduce patient dosing time, in one embodiment, it is important that liposomal entrapment or complexing of the lipid component to the copper chelator be highly efficient and that the lipid to copper chelator weight ratio be at as low a value as possible. In one embodiment, the weight ratio of the copper chelator to lipid component is from about 1.0 to 100.0 (1.0:100.0) to about 1.0 to 1.0 (1.0:1.0); from about 1.0 to 50.0 (1.0:50.0) to about 1.0 to 1.0 (1.0:1.0); from about 1.0 to 40.0 (1.0:40.0) to about 1.0 to 1.0 (1.0:1.0); from about 1.0 to 30.0 (1.0:30.0) to about 1.0 to 1.0 (1.0:1.0); from about 1.0 to 20.0 (1.0:20.0) to about 1.0 to 1.0 (1.0:1.0); from about 1.0 to 10.0 (1.0:10.0) to about 1.0 to 1.0 (1.0:1.0).

In another embodiment, the weight ratio of the copper chelator to lipid component is from about 1.0 to 50.0 (1.0:50.0) to about 1.0 to 5.0 (1.0:5.0); from about 1.0 to 20.0 (1.0:20.0) to about 1.0 to 5.0 (1.0:5.0); from about 1.0 to 15.0 (1.0:15.0) to about 1.0 to 5.0 (1.0:5.0); or from about 1.0 to 10.0 (1.0:10.0) to about 1.0 to 5.0 (1.0:5.0).

In one embodiment, the pharmaceutical composition provided herein comprises at least one copper chelator, a phospholipid and a sterol (e.g., cholesterol). In a further embodiment, the pharmaceutical composition comprises a copper chelator, DPPC and cholesterol.

In one embodiment, the copper chelator composition provided herein comprises the components provided in Table 2, below.

TABLE 2

| Composition # | Copper chelator | Lipid component |
|---|---|---|
| 1. | Bis-choline | DPPC, chol |
| | tetrathiomolybdate | |
| 2. | Bis-choline | DPPC, chol, DSPE |
| | tetrathiomolybdate | |
| 3. | Bis-choline | DPPC, chol, DPPG |
| | tetrathiomolybdate | |
| 4. | Bis-choline | DPPC, chol, DPPS |
| | tetrathiomolybdate | |
| 5. | Bis-choline | DPPC, chol, DPPG, DPPS |
| | tetrathiomolybdate | |
| 6. | Bis-choline | DPPC, chol, DHP |
| | tetrathiomolybdate | |
| 7. | Bis-choline | DPPC, chol, polyethylene glycol |
| | tetrathiomolybdate | hexadecyl ether (Brij ® 52) |
| 8. | Bis-choline | Chol, palmitic acid |
| | tetrathiomolybdate | |
| 9. | Bis-choline | Chol, DPPG, DPPS |
| | tetrathiomolybdate | |
| 10. | TTM ammonium | DPPC, chol |
| 11. | TTM ammonium | DPPC, chol, DSPE |
| 12. | TTM ammonium | DPPC, chol, DPPG |
| 13. | TTM ammonium | DPPC, chol, DPPS |
| 14. | TTM ammonium | DPPC, chol, DPPG, DPPS |
| 15. | TTM ammonium | DPPC, chol, DHP |
| 16. | TTM ammonium | DPPC, chol, polyethylene glycol |
| | | hexadecyl ether (Brij ® 52) |
| 17. | TTM ammonium | Chol, palmitic acid |
| 18. | TTM ammonium | Chol, DPPG, DPPS |
| 19. | TTM sodium | DPPC, Chol |
| 20. | TTM sodium | DPPC, chol, DSPE |
| 21. | TTM sodium | DPPC, chol, DPPG |
| 22. | TTM sodium | DPPC, chol, DPPS |
| 23. | TTM sodium | DPPC, chol, DPPG, DPPS |
| 24. | TTM sodium | DPPC, chol, DHP |
| 25. | TTM sodium | DPPC, chol, polyethylene glycol |
| | | hexadecyl ether (Brij ® 52) |
| 26. | TTM sodium | Chol, palmitic acid |
| 27. | TTM sodium | Chol, DPPG, DPPS |

Chol: cholesterol
DHP: dihexadecylphosphate
DPPC: dipalmitoylphosphatidylcholine
DPPG: dipalmitoylphosphatidylgycerol
DPPS: dipalmitoylphosphatidylserine

As described above, the composition in one embodiment includes lipid microparticles, lipid nanoparticles, liposomes or a combination thereof. The composition in one embodiment, comprises microparticles or nanoparticles comprising one or more of the copper chelators as described herein complexed to a lipid component, and a hydrophobic additive. In one embodiment, the hydrophobic additive (e.g., an additive that is at least partially hydrophobic) is a hydrocarbon, a terpene compound or a hydrophobic lipid (e.g., tocopherol, tocopherol acetate, sterol, sterol ester, alkyl ester, vitamin A acetate, a triglyceride, a phospholipid). The hydrocarbon can be aromatic, an alkane, alkene, cycloalkane or an alkyne. In one embodiment, the hydrocarbon is an alkane (i.e., a saturated hydrocarbon), in another embodiment, the hydrocarbon is a C₁₅-C₅₀ hydrocarbon. In a further embodiment, the hydrocarbon is a C₁₅, C₂₀, C₂₅, C₃₀, C₃₅, C₄₀, C₄₅ or C₅₀ hydrocarbon. In yet another embodiment, the hydrophobic additive is a C₁₅-C₂₅ hydrocarbon, C₁₅-C₃₅ hydrocarbon, C₁₅-C₄₅ hydrocarbon, C₁₅-C₂₀ hydrocarbon, C₂₀-C₂₅ hydrocarbon, C₂₅-C₃₀ hydrocarbon, C₃₀-C₃₅ hydrocarbon, C₃₅-C₄₀ hydrocarbon, C₄₀-C₄₅ hydrocarbon or a C₄₅-C₅₀ hydrocarbon.

The hydrophobic additive, when present in the composition, in one embodiment, is present at 25 mol %-50 mol %, for example, 30 mol %-50 mol %, 35 mol %-45 mol %. In even a further embodiment, the hydrophobic additive is present in the composition at about 40 mol % or about 45 mol %.

In one embodiment, a composition comprising a copper chelator compound, a lipid component, and a terpene compound (e.g., the hydrophobic additive) is provided. The composition, in a further embodiment, comprises a cationic lipid, e.g., a PEGylated cationic lipid, as the lipid component. The terpene compound (hydrophobic additive), in one embodiment, is a hydrocarbon (e.g., isoprene, squalene or squalene). In another embodiment, the terpene compound is a hemiterpene (C₅H₅), monoterpene (C₁₀H₁₆), sesquiterpene (C₁₅H₂₄), diterpene (C₂₀H₃₂) (e.g., cafestol, kahweol, cembrene, taxadiene), sesterterpene (C₂₅H₄₀), triterpene (C₃₀H₄₈), sesquaterpene (C₃₅H₅₆), tetraterpene (C₄₀H₆₄), polyterpene (e.g., a polyisoprene with trans double bonds) or a norisoprenoid (e.g., 3-oxo-α-ionol, 7,8-dihydroionone derivatives). The terpene compound, in another embodiment, is selected from one of the compounds provided in Table 3, below. In one embodiment, the hydrophobic additive is squalane.

TABLE 3
Terpene hydrophobic additives amenable for use in the compositions of
the present invention.

| Name | Formula |
|---|---|
| Isoprene | 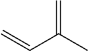 |
| Limonene | 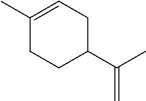 |
| humulene | 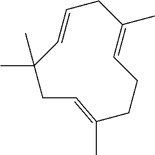 |
| farnasene | 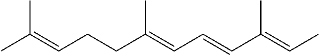 |
| squalene | 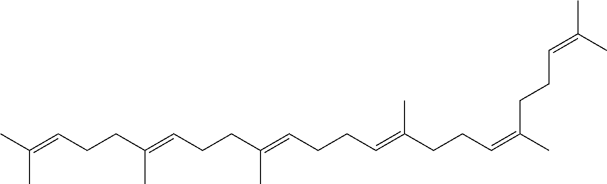 [SMILES: CC(C)=CCC/C(C)=C\CC/C(C)=C/CC/C=C(\C)CC/C=C(\C)CCC=C(C)C] |
| squalane | 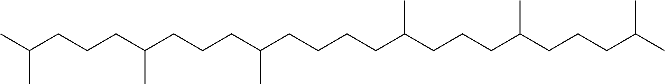 [SMILES: CC(C)CCCC(C)CCCC(C)CCCCC(C)CCCC(C)CCCC(C)C] |

The composition provided herein, in one embodiment, comprises a copper chelator and one or more PEGylated lipids. In a further embodiment, the composition comprises a hydrophobic additive, as described above. In one embodiment, the composition provided herein comprises a copper chelator, a hydrophobic additive and a PEGylated lipid. In a further embodiment, the hydrophobic additive comprises a hydrocarbon e.g, a terpene compound.

The present invention in another aspect provides a method for treating a subject for a vasculopathy. For example, the vasculopathy in one embodiment is pulmonary hypertension (e.g., pulmonary arterial hypertension (PAH) or portopulmonary hypertension (PPH)), peripheral vascular disease (PVD), ischemic lesions (e.g., lesions from critical limb ischemic (CLI)), coronary artery disease, post-angioplasty coronary artery restenosis, and diabetic vasculopathy. The term “subject” as used herein, refers to an animal, for example a mammal. The term “mammal” includes humans and both domestic animals such as laboratory animals and household pets (e.g., cats, dogs, swine, cattle, sheep, goats, horses, rabbits), and non-domestic animals such as wildlife and the like. In one embodiment, the subject is a human. Non-limiting examples of subjects treatable with the methods, compositions and kits described herein include a human, primate, cow, horse, sheep, goat, dog, cat, rabbit and a rodent. The term “subject” may be interchangeably used with the term patient in the context of the present invention.

In one embodiment, the subject is a patient who was non-responsive to a previous treatment, for example a PAH patient previously non-responsive to previous therapy.

The terms “treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

The method comprises in one embodiment, administering to a subject in need thereof a composition comprising an effective amount of one of the copper chelators described herein. Administration in one embodiment is via inhalation, oral, nasal, subcutaneous, transdermal, intraperitoneal or intravenous administration. It is understood that reference to a copper chelator compound in a treatment method also includes the use of an isomer, solvate, hydrate, hydrolysis product or pharmaceutically acceptable salt of the copper chelator.

The administration occurs, in one embodiment, once daily, twice daily, three times daily, every other day or once weekly.

For example, in one embodiment, the method for treating the vasculopathy (e.g, pulmonary hypertension such as PAH or PPH) comprises administering the the subject in need thereof a composition comprising an effective amount of a compound of Formula (I) and/or (II), a deprotonated anion, isomer, deutrated analog, solvate, hydrate, hydrolysis product, or a pharmaceutically acceptable salt thereof. Routes of administration to the patient include pulmonary (inhalation), subcutaneous, oral, nasal, intraperitoneal (IP), and the intravenous (IV) route.

In another embodiment, administration is via the oral route.

In another embodiment, administration is via the intravenous (IV) route.

In yet another embodiment, administration is via the pulmonary route via inhalation.

In another embodiment, administration is via intraperitoneal (IP) route. In some embodiment, administration is via intraperitoneal injection.

In even another embodiment, a composition of the invention is administered to a subject in need thereof via the IP route.

In one embodiment, the vasculopathy is pulmonary hypertension (PH). The World Health Organization (WHO) has classified PH into five groups. WHO Group I PH includes pulmonary arterial hypertension (PAH), idiopathic pulmonary arterial hypertension (IPAH), familial pulmonary arterial hypertension (FPAH), and pulmonary arterial hypertension associated with other diseases (APAH). For example, pulmonary arterial hypertension associated with collagen vascular disease (e.g., scleroderma), congenital shunts between the systemic and pulmonary circulation, portal hypertension and/or HIV infection are included in WHO Group I PH. The methods and compositions provided herein, in one embodiment, are provided to treat a WHO Group I PH patient in need thereof, for example a PAH patient, an IPAH patient, a FPAH patient or an APAH patient.

In one embodiment, the subject treated via a composition and/or method provided herein is a PAH patient. In another embodiment, the subject is a chronic thromboembolic pulmonary hypertension patient.

WHO Group II PH includes pulmonary hypertension associated with left heart disease, e.g., atrial or ventricular disease, or valvular disease (e.g., mitral stenosis). The methods and compositions provided herein, in one embodiment, are provided to treat a WHO Group II patient in need thereof.

WHO group III pulmonary hypertension is characterized as pulmonary hypertension associated with lung diseases, e.g., chronic obstructive pulmonary disease (COPD), interstitial lung disease (ILD), and/or hypoxemia. The methods provided herein, in one embodiment, are provided to treat a WHO Group III patient in need thereof.

WHO Group IV pulmonary hypertension is pulmonary hypertension due to chronic thrombotic and/or embolic disease. Group IV PH is also referred to as chronic thromboembolic pulmonary hypertension. Group IV PH patients experience blocked or narrowed blood vessels due to blood clots. The methods provided herein, in one embodiment, are provided to treat a WHO Group IV patient in need thereof.

WHO categorizes Group V PH as the “miscellaneous” category, and includes PH caused by blood disorders (e.g., polycythemia vera, essential thrombocythemia), systemic disorders (e.g., sarcoidosis, vasculitis) and/or metabolic disorders (e.g., thyroid disease, glycogen storage disease). The methods provided herein, in one embodiment, are provided to treat a WHO Group V patient in need thereof.

The New York Heart Association (NYHA) has categorized PAH patients into four functional classes, used to rate the severity of the disease. Class I PAH patients as categorized by the NYHA, do not have a limitation of physical activity, as ordinary physical activity does not cause undue dyspnoea or fatigue, chest pain, or near syncope. Class II PAH patients as categorized by the NYHA have a slight limitation on physical activity. These patients are comfortable at rest, but ordinary physical activity causes undue dyspnoea or fatigue, chest pain or near syncope. Class III PAH patients as categorized by the NYHA have a marked limitation of physical activity. Although comfortable at rest, class III PAH patients experience undue dyspnoea or fatigue, chest pain or near syncope as a result of less than ordinary physical activity. Class IV PAH patients as categorized by the NYHA are unable to carry out any physical activity without symptoms. Class IV PAH patients might experience dyspnoea and/or fatigue at rest, and discomfort is increased by any physical activity. Signs of right heart failure are often manifested by class IV PAH patients. The methods provided herein, in one embodiment, are provided to treat an NYHA Class I, II, III or IV PAH patient in need thereof. For example, the NYHA Class I, II, III or IV PAH patient is administered a composition comprising administering a composition comprising an effective amount of a copper chelator, deprotonated anion, isomer, deutrated analog, solvate, hydrate, hydrolysis product or a pharmaceutically acceptable salt thereof. Administration is via a pulmonary (inhalation), a subcutaneous, oral, nasal, intraperitoneal or an intravenous route.

Portopulmonary hypertension is defined by the coexistence of portal and pulmonary hypertension, and is a serious complication of liver disease. The diagnosis of portopulmonary hypertension is based on hemodynamic criteria: (1) portal hypertension and/or liver disease (clinical diagnosis-ascites/varices/splenomegaly), (2) mean pulmonary artery pressure >25 mmHg at rest, (3) pulmonary vascular resistance >240 dynes s/cm⁵, (4) pulmonary artery occlusion pressure <15 mmHg or transpulmonary gradient >12 mmHg. PPH is a serious complication of liver disease, and is present in 0.25 to 4% of patients suffering from cirrhosis. Today, PPH is comorbid in 4-6% of those referred for a liver transplant.

In one embodiment, the subject in need of treatment is a portopulmonary hypertension patient. Accordingly, in this embodiment, the vasculopathy is portopulmonary hypertension (PPH), In one embodiment, the method comprises administering an effective amount of one of the compositions described herein (i.e., a composition comprising an effective amount of a copper chelator, deprotonated anion, isomer, deutrated analog, solvate, hydrate, hydrolysis product or a pharmaceutically acceptable salt thereof), via a pulmonary (inhalation), a subcutaneous, oral, nasal, intraperitoneal or an intravenous route of administration, to a patient in need of PPH treatment.

In one embodiment, the subject in need of treatment suffers from a peripheral vascular disease. Accordingly in this embodiment, a method for treating peripheral vascular disease via administration to the subject of one of the copper chelators compositions is provided. The peripheral vascular disease in one embodiment is peripheral arterial occlusive disease or intermittent claudication. In one embodiment, the method comprises administering an effective amount of one of the compositions described herein (i.e., a composition comprising an effective amount of a copper chelator, deprotonated anion, isomer, deutrated analog, solvate, hydrate, hydrolysis product or a pharmaceutically acceptable salt thereof), via a pulmonary (inhalation), a subcutaneous, oral, nasal, intraperitoneal or an intravenous route of administration, to the subject in need of peripheral vascular disease treatment.

Coronary artery disease (atherosclerosis) is a progressive disease in humans where one or more coronary arteries gradually become occluded through the buildup of plaque. In one embodiment, a patient in need of coronary artery disease is treated with one of the compositions provided herein. In one embodiment, a method for treating coronary artery disease is provided comprising administering to a patient in need thereof a composition comprising an effective amount of a copper chelator, e.g., a copper chelator of Formula I or II, deprotonated anion, isomer, deutrated analog, solvate, hydrate, hydrolysis product or a pharmaceutically acceptable salt thereof. In a further embodiment, administration is via pulmonary (inhalation), subcutaneous, oral, nasal, intracoronary, intraperitoneal or an intravenous route.

In one embodiment, the subject in need of treatment is a diabetic vasculopathy patient. In this embodiment, a method for treating diabetic vasculopathy via administration to the subject of one of the copper chelators compositions is provided. In one embodiment, the method comprises administering an effective amount of one of the compositions described herein (i.e., a composition comprising an effective amount of a copper chelator, deprotonated anion, isomer, deutrated analog, solvate, hydrate, hydrolysis product or a pharmaceutically acceptable salt thereof), via a pulmonary (inhalation), a subcutaneous, oral, nasal, intraperitoneal or an intravenous route of administration, to the subject in need of diabetic vasculopathy treatment.

In one embodiment, the subject in need of treatment has an ischemic lesion. Accordingly in one embodiment, the method comprises administering an effective amount of one of the compositions described herein (i.e., a composition comprising an effective amount of a copper chelator, deprotonated anion, isomer, deutrated analog, solvate, hydrate, hydrolysis product or a pharmaceutically acceptable salt thereof), via a pulmonary (inhalation), a subcutaneous, oral, nasal, intraperitoneal or an intravenous route of administration, to the subject in need of ischemic lesion treatment.

The ischemic lesion in one embodiment is a digital ischemic lesion, such as a digital ulcer or a necrotic lesion. The method for treating the digital ischemic lesion in one embodiment ameliorates a symptom or functional deficit and/or reduces the number of symptoms and/or functional deficit(s) associated with a digital ischemic lesion. The term “digital ischemic lesion” refers to a lesion on a digit, i.e., a toe or a finger, of a subject, such as a human. In one embodiment, the digital ischemic lesion may be caused by or associated with an ischemic disease or condition, such as scleroderma, including systemic sclerosis, or Raynaud's Phenomenon. The symptom that may be ameliorated and/or reduced may be, for example, a pain associated with a digital ischemic ulcer and/or scleroderma. In some embodiments, administering a copper chelator composition provided herein, upon administration to a patient in need of treatment, provides amelioration or reduction of one or more functional deficits associated with a digital ischemic lesion. For example, in one embodiment, the copper chelator composition provided herein ameliorates or reduces a hand function deficit, i.e., provides an improvement in the hand function of the treated patient. Administration, in one embodiment, is via inhalation (e.g., with a nebulizer or MDI), oral, nasal, subcutaneous, transdermal, intraperitoneal or intravenous administration.

In one embodiment, the ischemic lesion is due to critical limb ischemia (CLI). CLI is a severe obstruction of the arteries which markedly reduces blood flow to the extremities (hands, feet and legs) and has progressed to the point of severe pain and ischemic lesions. CLI is the advanced stage of peripheral artery disease (PAD), which results from a progressive thickening of artery lining (caused by a buildup of plaque). This buildup of plaque, also known as atherosclerosis, narrows or blocks blood flow, reducing circulation of blood to the legs, feet or hands. The risk factors for critical limb ischemia include age, smoking status, diabetes, obesity, high cholesterol, high blood pressure, sedentary lifestyle, family history of vascular disease.

As provided herein, methods for treating a vasculopathy include administering to a subject in need thereof a composition comprising an effective amount of one of the copper chelators described herein. Administration in one embodiment is via inhalation, oral, nasal, subcutaneous, transdermal, intraperitoneal or intravenous administration.

In one embodiment, a composition of the present invention is administered to a patient in need thereof via continuous intravenous or continuous subcutaneous infusion, e.g., via an infusion pump. The patient in one embodiment is a WHO Group I PAH, for example, to diminish symptoms associated with exercise in a patient in need thereof, or to increase exercise capacity. In another embodiment, the PAH patient is a NYHA class I, NYHA class II NYHA class III or NYHA class IV patient. In even another embodiment, the PAH is associated with congenital systemic-to-pulmonary shunts or PAH associated with connective tissue diseases.

In one embodiment, subcutaneous infusion delivers a copper chelator composition just beneath the surface of the skin.

In one embodiment, an infusion device continuously infuses a copper dictator composition subcutaneously for a predetermined interval, the predetermined interval may be at or about 1 hour, 2 hours, 3 hours, 4, hours, 5, hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours 72 hours, 76 hours, 80 hours, 84 hours, 88 hours, 92 hours, or 96 hours.

In one embodiment, the present invention encompasses a subcutaneous infusion device to deliver one or more of the copper chelator compositions described herein. Subcutaneous infusion devices provide an ease of use in delivering pharmaceutical compositions to patients that would otherwise require repeated penetration of the integument to deliver pharmaceutical compositions throughout a short period of time. The use of subcutaneous infusion devices further provide a greater degree of mobility as compared to patients that rely upon an intravenous (IV) drip system for drug delivery. An advantage of subcutaneous infusion over other delivery methods is that blood plasma levels of a drug are considerably more stable, and appropriate symptom control can be achieved without the potentially toxic effects of the peaks and troughs resulting from episodic drug administration. The use of subcutaneous infusion allows for a continuous infusion of the copper chelator over a calculated period of time and can provide constant dosing of the copper chelator composition.

An infusion pump provided herein is designed for subcutaneous infusion (e.g., continuous subcutaneous infusion) and/or intravenous infusion (e.g., continuous intravenous infusion). The pump in one embodiment, is small and lightweight, adjustable to provide different programmable infusion rates, comprises one or more alarms to monitor occlusion, delivery progress, low battery, programming error and motor malfunction. In one embodiment, the infusion pump comprises a drug reservoir. In a further embodiment, the reservoir comprises one of the copper chelator compositions provided herein. In a further embodiment, the device comprises a monitor to monitor the dosage of delivered copper chelator.

The infusion pump provided herein, in one embodiment, is ambulatory, has a delivery accuracy of ±6% or better and is positive pressure driven. In a further embodiment, the pump comprises a reservoir and the reservoir is made of polyvinyl chloride, polypropylene or glass.

In another embodiment, the infusion pump (subcutaneous or intravenous) comprises a pump, a reservoir containing the copper chelator composition, an infusion set for subcutaneous infusion of the composition, and an optional monitor mea ruing concentration of copper dictators or metabolite(s) thereof. In another embodiment, the infusion device provides an open-loop or closed-loop system.

The term “closed-loop system,” as used herein, refers to an integrated system for providing an infusion of a composition. Closed-loop systems contain a mechanism for measuring prostacyclins, or metabolites thereof, a mechanism for delivering one or more compositions, and a mechanism for determining the amount of the one or more compositions needed to be delivered to achieve desired results. A closed-loop system may contain a copper chelator sensor, a copper chelator composition delivery device, such as a pump or infuser, and a controller that receives information from the sensor and provides commands to the delivery device. The commands can be generated by software in the controller. The software may include an algorithm to determine the amount of a prostacyclin composition to be delivered, based upon the prostacyclin detected by the prostacyclin sensor or anticipated by the user.

The term “open-loop system,” as used herein, refers to devices similar to a closed-loop system, except that open-loop system devices do no automatically measure and respond to copper chelator composition levels. In an open-loop system a pump, infuser, or other similar device is programmed to infuse a composition continuously, and where the patient is able, by means of a user input on the pump or other means to administer an increase or decrease of the one or more compositions.

In another embodiment the infusion device continuously infuses the copper chelator composition for a predetermined interval; wherein at the end of the predetermined interval the predetermined infusion interval may repeat or initiate a new predetermined infusion interval, in another embodiment, the predetermined interval is about 24 hours, about 36 hours, or less than about 96 hours.

In another embodiment, the subcutaneous infusion of the copper chelator composition occurs at either a continuous rate of volume or a variable rate of volume.

In one embodiment, a kit for the administration of a copper chelator composition described herein in amounts effective to treat a vasculopathy, e.g., pulmonary arterial hypertension. The kit comprises a composition comprising one of the copper chelators described herein, a subcutaneous infusion pump, and instructions for the administration of a copper chelator composition. In another embodiment, the subcutaneous infusion pump of the kit is a continuous subcutaneous infusion pump.

In one embodiment, the present invention encompasses an intravenous (IV) infusion in the delivery of one or more of the copper chelator compositions described herein. IV delivery can range from an intravenous infusion with or without an infusion pump, intravenous cannula with an injection port, or intravenous through a central venous line. IV delivery provides a direct rought to the bloodstream which allows for the administration of any number of compounds to be quickly disseminated by the circulatory system. In a further embodiment, the intravenous infusion may be carried out with a hypodermic needle which is connected to a syringe or a continuous drip reservoir (e.g., IV bag). In a further embodiment, the intravenous infusion is carried out with the insertion of a peripheral cannula or a central line. In a further embodiment, the intravenous infusion is carried out with infusion pump. The intravenous infusion can be performed intermittently or continuously.

Administration of the copper chelator composition to a patient in need thereof, in one embodiment, is via pulmonary administration. With respect to the pulmonary route, the copper chelators composition may be used in any dosage dispensing device adapted for such administration. The pulmonary administration and can be, for example, with an inhalation delivery device such as a metered dose inhaler (MDI), dry powder inhaled (DPI), soft mist inhaler, or a nebulizer. The inhalation delivery device can contain and be used to deliver a single dose of the copper chelator composition or the device can contain and be used to deliver multi-doses of the composition of the present invention. The device, in one embodiment, is constructed to ascertain optimum metering accuracy and compatibility of its constructive elements, such as container, valve and actuator with the formulation and could be based on a mechanical pump system, e.g., that of a metered-dose nebulizer, dry powder inhaler, soft mist inhaler, or a nebulizer. For example, pulmonary delivery devices include a jet nebulizer, electronic nebulizer, a soft mist inhaler, and a capsule-based dry powder inhaler.

In one embodiment, a metered dose inhalator (MDI) is employed as the inhalation delivery device for the compositions of the present invention. In a further embodiment, the copper chelators compound or composition of the invention is suspended in a propellant (e.g., hydrofluorocarbon) prior to loading into the MIA The basic structure of the MDI comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydrofluoroalkanes (HFAs) such as 134a and 227. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

In one embodiment, a composition of the invention is administered via a metered dose inhaler (MDI) to a patient in need of treatment. The composition or compound, in one embodiment, is delivered via a MDI by the use of a propellant, for example, a chlorofluorocarbon (CFC) or a fluorocarbon. In one embodiment, where delivery is via an MDI, the compound is suspended or dissolved directly in a propellant solution. The patient, in one embodiment, is administered the copper chelator composition of the invention once daily, twice daily or three times daily. In one embodiment, the administration is with food. In one embodiment, each administration comprises 1 to 5 doses (puffs) from an MDI, for example 1 dose (1 puff), 2 dose (2 puffs), 3 doses (3 puffs), 4 doses (4 puffs) or 5 doses (5 puffs). The MDI, in one embodiment, is small and transportable by the patient.

In another embodiment, the copper chelator composition is administered via a nebulizer to a patient in need of treatment of a vasculopathy, e.g., PAH. The administration occurs, in one embodiment, once daily or twice daily, three times daily, every other day or once weekly.

In one embodiment, a composition of the present invention is administered to a patient in need thereof via a dry powder inhaler (DPI) to a patient in need of treatment. The patient, in one embodiment, is administered the copper chelator composition of the invention once daily or twice daily. In one embodiment, the administration is with food. In one embodiment, each administration comprises 1 to 5 doses (puffs) from a DPI, for example 1 dose (1 puff), 2 dose (2 puffs), 3 doses (3 puffs), 4 doses (4 puffs) or 5 doses (5 puffs). The DPI, in one embodiment, is small and transportable by the patient.

The compositions of the present invention may be used in any dosage dispensing device adapted for pulmonary administration. Accordingly, in one aspect, the present invention provides systems comprising one or more of the compositions described herein and an inhalation delivery device. The device, in one embodiment, is constructed to ascertain optimum metering accuracy and compatibility of its constructive elements, such as container, valve and actuator with the composition and could be based on a mechanical pump system, e.g., that of a metered-dose nebulizer, dry powder inhaler, metered dose inhaler (MDI), soft mist inhaler, or a nebulizer. For example, inhalation delivery devices include a jet nebulizer, electronic nebulizer, a soft mist inhaler, and a capsule-based dry powder inhaler, all of which are amenable for use with the compositions of the present invention.

The composition, in one embodiment, is administered via a nebulizer, which provides an aerosol mist of the composition for delivery to the lungs of a subject. A nebulizer type inhalation delivery device can contain the compositions of the present invention as an aqueous solution or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the composition via an electrochemical vibrating surface. At a given amplitude the waveform becomes unstable, whereby it disintegrates the liquids film, and it produces small droplets of the composition. The nebulizer device driven by air or other gases operates on the basis that a high pressure gas stream produces a local pressure drop that draws the liquid composition into the stream of gases via capillary action. This fine liquid stream is then disintegrated by shear forces.

A nebulizer type inhalation delivery device can contain the compositions of the present invention as a solution, usually aqueous, or a suspension. For example, the composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®, the Omron MicroAir, the Nektar Aeroneb®, and the PARI eFlow®. Other devices that can be used with the compositions described herein include jet nebulizers (e.g., PARI LC Star, AKITA), soft mist inhalers, and capsule-based dry powder inhalers (e.g., PH&T Turbospin).

The nebulizer may be portable and hand held in design, and may be equipped with a self-contained electrical unit. The nebulizer device may comprise a nozzle that has two coincident outlet channels of defined aperture size through which the liquid composition can be accelerated. This results in impaction of the two streams and atomization of the composition. The nebulizer may use a mechanical actuator to force the liquid composition through a multiorifice nozzle of defined aperture size(s) to produce an aerosol of the composition for inhalation. In the design of single dose nebulizers, blister packs containing single doses of the composition may be employed.

The device can contain, and be used to deliver, a single dose of the compositions of the invention, or the device can contain, and be used to deliver, multi-doses of the compositions of the invention.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the composition from the device. Suitable propellants, e.g., for MDI delivery, may be selected among such gases as fluorocarbons, chlorofluorocarbons (CFCs), hydrocarbons, hydrofluorocarbons, hydrofluoroalkane propellants (e.g., HFA-134a and HFA-227), nitrogen and dinitrogen oxide or mixtures thereof.

In one embodiment, a propellant is present in a composition intended for MDI delivery, and is selected from a fluorocarbon, chlorofluorocarbon (GEC), hydrocarbons, hydrofluoroalkane propellants (e.g., HFA-134a and HFA-227), nitrogen and dinitrogen oxide or mixtures thereof. In embodiments of the present invention, the propellant is CFC-12 or an ozone-friendly, non-CFC propellant, such as 1,1,1,2-tetrafluoroethane (HFC 134a), 1,1,1,2,3,3,3-heptafluoropropane (HFA-227), HCFC-22 (difluorochloromethane), HFA-152 (difluoroethane and isobutene), trans-1,3,3,3,-tetrafluoropro-1-ene (HFO 1234ze) and 2,3,3,3,-tetrafluoroprop-1-ene (HFO 1234yf), or combinations thereof.

The composition may consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. The inhalation delivery device, in one embodiment, delivers a single dose via, e.g., a blister pack, or it may be multi dose in design. The pressurized metered dose inhalator of the inhalation system can be breath actuated to deliver an accurate dose of the composition. To insure accuracy of dosing, the delivery of the composition may be programmed via a microprocessor to occur at a certain point in the inhalation cycle. The MDI may be portable and hand held.

Upon aerosolization, the aerosolized composition is in the form of aerosolized particles. The aerosolized composition can be characterized by the particle size of the aerosol, for example, by measuring the “mass median aerodynamic diameter” or “fine particle fraction” associated with the aerosolized composition. “Mass median aerodynamic diameter” or “MMAD” is normalized regarding the aerodynamic separation of aqua aerosol droplets and is determined by impactor measurements, e.g., the Anderson Cascade Impactor (ACI) or the Next Generation Impactor (NGI). The gas flow rate, in one embodiment, is 28 Liter per minute for the ACI and 15 liter per minute for the NGI.

Yet another aspect of the invention relates to the compositions described above in aerosolized form. Upon nebulization or aerosolization, the aerosolized composition is in the form of aerosolized particles. The aerosolized composition can be characterized by the particle size of the aerosol, for example, by measuring the “mass median aerodynamic diameter” or “fine particle fraction” associated with the aerosolized composition. “Mass median aerodynamic diameter” or “MMAD” is normalized regarding the aerodynamic separation of aqua aerosol droplets and is determined by impactor measurements, e.g., the Anderson Cascade Impactor (ACI) or the Next Generation Impactor (NGI). The gas flow rate, in one embodiment, is 28 Liter per minute for the ACI and 15 liter per minute for the NGI.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the nebulized composition provided herein, in one embodiment is less than 5 μm or about 1 μm to about 5 μm, and has a GSD in a range of 1.0 to 2.2, or about 1.0 to about 2.2, or 1.5 to 2.2, or about 1.5 to about 2.2.

“Fine particle fraction” or “FPF,” as used herein, refers to the fraction of the aerosol having a particle size less than 5 μm in diameter, as measured by cascade impaction. FPF is usually expressed as a percentage

In the present invention as provided above, the nebulizer may be employed to ensure the sizing of particles is optimal for positioning of the particle within, for example, the pulmonary membrane.

In one embodiment, the mass median aerodynamic diameter (MMAD) of the aerosol particles is about 1 μm to about 5 μm, or about 1 μm to about 4 μm, or about 1 μm to about 3 μm, or about 2 μm to about 3 μm, or about 1 μm to about 2 μm, as measured by cascade impaction, for example, by the ACI or NGI.

In another embodiment, the MMAD of the aerosol particles is about 5 μm or less, about 4 μm or less, about 3 μm or less, about 2 μm or less, or about 1 μm or less, as measured by cascade impaction, for example, by the ACI or NGI.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the aerosolized composition provided herein, in one embodiment is less than 5 μm or about 1 μm to about 5 μm, and has a GSD in a range of from about 1.0 to about 2.2, or from about 1.5 to about 2.2, as measured by the ACI or NGI.

“Respirable mass” or “RM”, as used herein, is usually expressed as μg/shot and is the total amount of emitted drug product that exits the metered dose inhaler upon actuation.

In one embodiment, the respirable mass of the aerosol particles is about 1 μg/shot to about 100 μg/shot, or about 1 μg/shot to about 50 μg/shot, or about 1 μg/shot to about 40 μg/shot, or about 1 μg/shot to about 30 μg/shot, or about 3 μg/shot to about 80 μg/shot, or about 3 μg/shot to about 70 μg/shot, or about 3 μg/shot to about 60 μg/shot, about 3 μg/shot to about 50 ₁1g/shot, about 3 psi/shot to about 40 μg/shot, about 3 μg/shot to about 30 μg/shot, as measured by the ACI or NGI.

“Fine particle fraction” or “FPF”, as used herein, refers to the fraction of the aerosol having a particle size less than 5 μm in diameter, as measured by cascade impaction. FPF is usually expressed as a percentage.

In one embodiment, the fine particle fraction (FPF) of the aerosol particles is greater is greater than or equal to about 40%, is greater than or equal to about 50%, is greater than or equal to about 60%, is greater than or equal to about 70%, is greater than or equal to about 80%, greater than or equal to about 85%, greater than or equal to about 90%, or greater than or equal to about 95%, as measured by the ACI or NGI.

In another embodiment, the FPF of the aerosol particles is about 40% to about 99%, is about 50% to about 99%, is about 60% to about 99%, is about 70% to about 99%, is about 75% to about 99%, is about 80% to about 99%, is about 80% to about 95%, is about 80% to about 90%, or is about 85% to about 90%, or is about 85% to about 95%, as measured by the ACI or NGI.

“Percent throat deposition” or “PTD” is the amount of drug deposited on the throat of the cascade impactor and is expressed as a percentage.

In one embodiment, the percent throat deposition is less than or equal to about 60%, less than or equal to about 50%, less than or equal to about 40%, less than or equal to about 30%, less than or equal to about 25%, as measured by the ACI or NGI.

In one embodiment, a dry powder inhaler (DPI) is employed as the inhalation delivery device for the compositions of the present invention. In one embodiment, the DPI generates particles having an MMAD of from about 1 μm to about 10 μm, or about 1 μm to about 9 μm, or about 1 μm to about 8 μm, or about 1 μm to about 7 μm, or about 1 μm to about 6 μm, or about 1 μm to about 5 μm, or about 1 μm to about 4 μm, or about 1 μm to about 3 μm, or about 1 μm to about 2 μm in diameter, as measured by the NGI or ACI. In another embodiment, the DPI generates a particles having an MMAD of from about 1 μm to about 10 μm, or about 2 μm to about 10 μm, or about 3 μm to about 10 μm, or about 4 μm to about 10 μm, or about 5 μm to about 10 μm, or about 6 μm to about 10 μm, or about 7 μm to about 10 μm, or about 8 μm to about 10 μm, or about 9 μm to about 10 μm, as measured by the NGI or ACI.

In one embodiment, the MMAD of the particles generated by the DPI is about 10 um or less, about 9 μm or less, about 8 μm or less, about 7 μm. or less, about 6 μm or less, about 5 μm or less, about 4 μm or less, about 3 μm or less, about 2 μm or less, or about 1 μm or less, as measured by the NGI or ACI.

In one embodiment, the MMAD of the particles generated by the DPI is less than about 9.9 μm, less than about 9.5 μm, less than about 9.3 μm, less than about 9.2 μm, less than about 9.1 μm, less than about 9.0 μm, less than about 8.5 μm, less than about 8.3 μm, less than about 8.2 μm, less than about 8.1 μm, less than about 8.0 μm, less than about 7.5 μm, less than about 7.3 μm, less than about 7.2 μm, less than about 7.1 μm, less than about 7.0 μm, less than about 6.5 μm, less than about 6.3 μm, less than about 6.2 μm, less than about 6.1 μm, less than about 6.0 μm, less than about 5.5 μm, less than about 5.3 μm, less than about 5.2 μm, less than about 5.1 μm, less than about 5.0 μm, less than about 4.5 μm, less than about 4.3 μm, less than about 4.2 μm, less than about 4.1 μm, less than about 4.0 μm or less than about 3.5 μm, as measured by the NGI or ACI.

In one embodiment, the MMAD of the particles generated by the DPI is from about 1.0 μm to about 10.0 μm, from about 2.0 μm to about 9.5 μm, from about 2.5 μm to about 9.0 μm, from about 3.0 μm to about 9.0 μm, from about 3.5 μm to about 8.5 μm or from about 4.0 μm to about 8.0 μm.

In one embodiment, the FPF of the copper chelator composition generated by the DPI is greater than or equal to about 40%, greater than or equal to about 50%, greater than or equal to about 60%, or greater than or equal to about 70%, as measured by the ACI or NGI. In another embodiment, the FPF of the aerosolized composition is about 80% to about 99%, about 80% to about 95%, about 80% to about 90%, or about 85% to about 90%, or about 85% to about 95%, as measured by the NGI or ACI.

The copper chelator composition, in one aspect of the invention, is packaged as a kit that further includes an inhalation delivery device, a subcutaneous infusion pump, an intravenous infusion pump or a transdermal patch delivery system. The inhalation device may be disposable, single-use or a multiple-use device. In another embodiment, the inhalation device comprises a metered dose inhaler (MDI), a dry powder inhaler (DPI) or a nebulizer. In one embodiment, the copper chelator is TTM, a hydrolysis product thereof or a pharmaceutically acceptable salt thereof.

The devices and/or compositions described here may be packaged and/or distributed (e.g., to hospitals, clinics, physicians, and/or patients) in an administration kit. Such kits may comprise one or more inhalation devices (e.g., MDI, DPI or nebulizer), and one or more containers (e.g., unit doses or multi-dose containers) of the composition. In some variations, the kit may include one or more devices that are already loaded with the composition. For example, a device may comprise a reservoir that is pre-filled with the composition. Certain variations of kits may include multiple different compositions, and/or multiple different dosages of the same composition. The kit may additionally comprise a carrier or diluent, a case, and/or instructions for operating the appropriate device.

In one embodiment, a copper chelator composition provided herein is administered in combination with one or more additional active agents.

In some embodiments, such one or more additional active agents can be also administered in the same composition as the copper chelator. In one embodiment, such one or more additional active agents can be administered separately, i.e., prior to, or subsequent to, the copper chelator compound or composition provided herein. Particular additional active agents that can be administered in combination with the copper chelator may depend the particular treatment method and disorder to be treated. In some cases, the additional active agent can be a cardiovascular agent such as a cox-2 inhibitor, a rho kinase inhibitor, a calcium channel blocker, a phosphodiesterase inhibitor, an endothelial antagonist, or an anti platelet agent.

In one embodiment, one or more additional active agent is a prostacyclin analog such as treprostinil, iloprost or cisaprost. In a further embodiment, the one or more additional active agents is treprostinil or a prodrug thereof, e.g., an alkyl ester prodrug.

### EXAMPLES

The present invention is further illustrated by reference to the following Examples. However, it should be noted that these Examples, like the embodiments described above, are illustrative and are not to be construed as restricting the scope of the invention in any way.

### Example 1

Liposomal TTM preparation

Lipid, 50 mg DPPC/DPPG/Chol was added to a glass vial at a molar ratio 60:10:30 mol %, dried under stream of N₂, dissolved in tert-butanol, frozen and lyophilized to obtain dry cake.

Lipid cake was then hydrated by adding 2 mL of solution comprised of TTM ammonium salt (20 mM), sodium borate (10 mM), and the pH was adjusted to 9.0 by adding sodium hydroxide.

The resulting mixture was incubated for at least 1 hr. or more at room temperature. The mixture was vortexed periodically until a homogeneous suspension was formed.

Optionally, the suspension is subjected to a freeze-thaw cycle (1×, 2× or 3×).

The suspension is passed through a 400 nm membrane 5 times. Next, the suspension is passed through a 200 nm membrane 10 times.

Unencapsulated TTM is removed via a G25 Minitrap or PD-10 column pre-equilibrated with osmotically balanced wash buffer.

Liposomes are characterized by particle size, TTM content and lipid concentration.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1-218. (canceled)

219. A method for treating a vasculopathy in a patient in need thereof, comprising administering to the patient a composition comprising an effective amount of a copper chelator compound.

220. The method of claim 219, wherein the copper chelator compound comprises tetrathiomolybdate (TTM).

221. The method of claim 219, wherein the vasculopathy is selected from the group consisting of pulmonary hypertension, peripheral vascular disease, ischemic lesions, coronary artery disease and diabetic vasculopathy.

222. The method of claim 219, wherein the vasculopathy is pulmonary arterial hypertension (PAH) or portopulmonary hypertension (PPH).

223. The method of claim 222, wherein the vasculopathy is a pulmonary arterial hypertension (PAH).

224. The method of claim 220, wherein the copper chelator compound is bis-choline tetrathiomolybdate.

225. The method of claim 220, wherein the compound is a compound of the formula XY wherein X is (NH₄)(NH₄) and Y is MoS₄.

226. The method of claim 219, wherein the composition comprises a pharmaceutically acceptable carrier, diluent and/or excipient.

227. The method of claim 219, wherein the composition is formulated as a dry powder, solution or suspension.

228. The method of claim 219, wherein the composition is administered via oral, subcutaneous, transdermal, intraperitoneal or intravenous administration.

229. The method of claim 228, wherein the composition is administered via oral administration.

230. The method of claim 228, wherein the composition is administered via subcutaneous administration.

231. The method of claim 228, wherein the composition is administered via transdermal administration.

232. The method of claim 228, wherein the composition is administered via intraperitoneal administration.

233. The method of claim 228, wherein the composition is administered via intravenous administration.

234. The method of claim 221, wherein the vasculopathy is peripheral vascular disease.

235. The method of claim 221, wherein the vasculopathy is peripheral ischemic lesions.

236. The method of claim 221, wherein the vasculopathy is peripheral coronary artery disease.

237. The method of claim 221, wherein the vasculopathy is diabetic vasculopathy.

238. The method of claim 219, wherein the vasculopathy is portopulmonary hypertension (PPH).
